(19) United States
(12) Patent Application Publication
(10) Pub. No.: US 2010/0092963 A1
(43) Pub. Date: Apr. 15, 2010

(54) KITS AND METHODS FOR SELECTIVE AMPLIFICATION AND DETECTION OF NUCLEIC ACID TARGET

(76) Inventor: Jingliang JU, San Diego, CA (US)

Correspondence Address: JONES DAY, 222 EAST 41ST ST, NEW YORK, NY, 10017, US

(21) Appl. No.: 12/485,035

(22) Filed: Jun. 15, 2009

Related U.S. Application Data
(60) Provisional application No. 61/131,979, filed on Jun. 13, 2008.

(51) Int. Cl. C12Q 1/68

(52) U.S. Cl. 435/6

(57) ABSTRACT

The application relates generally to kits and methods useful for the selective capture, amplification and/or detection of one or more nucleic acid targets, as well as compositions comprising said amplification reaction mixtures. More specifically, the application relates to a signal primer that comprises (i) a target-specific sequence which hybridizes specifically to a nucleic acid target, and (ii) a signal sequence upstream of the target-specific sequence, wherein the signal sequence is preferably not found in the nucleic acid target or its complementary sequence; and a detection means for detecting the presence of the complementary sequence of the signal sequence in amplified nucleic acid target products.

The application claims the benefit of U.S. Provisional Application No. 61/131,979, filed Jun. 13, 2008, the content of which is hereby incorporated by reference in its entirety.

### 1. FIELD OF THE INVENTION

The application relates generally to kits and methods useful for the selective capture, amplification and/or detection of one or more nucleic acid targets, as well as compositions comprising said amplification reaction mixtures. More specifically, the application relates to a signal primer that comprises (i) a target-specific sequence which hybridizes specifically to a nucleic acid target, and (ii) a signal sequence upstream of the target-specific sequence; and a detection means for detecting the presence of the complementary sequence of the signal sequence in amplified nucleic acid target products.

### 2. BACKGROUND

Real-time polymerase chain reaction (PCR) or transcription-mediated amplification (TMA) assays are currently used as diagnostic tools in clinical applications (see, e g., Ian M. MacKay, “Real-Time PCR in Microbiology: From Diagnosis to Characterization,” Caister Academic Press, September 2007, ISBN: 978-1-904455-18-9; and “Current Protocols in Molecular Biology,” Last Update: May 14, 2009, Print ISSN: 1934-3639, Online ISSN: 1934-3647). The real-time PCR and TMA assays are carried out in the closed-tube format and can be used to obtain quantitative results. The chemistry of real-time PCR and TMA assays are based on monitoring fluorescence at temperatures that facilitate calculating the kinetics of the product formed and performing melting curve analyses to identify formation of the specific product. Fluorescence is usually monitored using an optical device to collect the data at specific excitation and emission wavelengths for the particular fluorescent dye present in the sample. The design of the specific primers and specific molecular beacons and other target-specific probes is a time-consuming step, which requires specific software and experience.

One method used to monitor nucleic acid amplification is the addition of SY BR Green I dye (Ririe et al., Anal. Biochem. 245:154-60, 1997) and LCGreen (Wittwer et al., Clin. Chem. 49:853-60, 2003) during PCR. During amplification, these fluorophores are excited with the appropriate wavelength of light, inducing fluorescence when the dye intercalates into a DNA double helix. However, this method lacks specificity and the primer-dimer can also fluoresce.

Specificity can be increased by using a labeled sequence-specific probe. Several of such methods are currently available for performing real-time PCR, such as TaqMan probes (Lee et al., Nucleic Acids Res. 21:3761-6, 1993); molecular beacons (Tyagi and Kramer, Nat. Biotechnol. 14:303-8, 1996); self-probing amplicons (scorpions) (Whitcombe et al., Nat. Biotechnol. 17:804-7, 1999); Amplisensor (Chen et al., Appl. Environ. Microbiol. 64:4210-6, 1998); Amplifluor (Nazarenko et al., Nucleic Acids Res. 25:2516-21, 1997 and U.S. Pat. No. 6,117,635); displacement hybridization probes (Li et al., Nucleic Acids Res. 30:E5, 2002); fluorescent restriction enzyme detection (Cairns et al., Biochem. Biophys. Res. Commun. 318:684-90, 2004); and adjacent hybridization probes (Wittwer et al., Biotechniques 22:130-1, 134-8, 1997). Todd et al. described a method using a zymogene which encodes, but which itself is the anti-sense sequence of, a catalytic nucleic acid sequence (Todd et al., Clin. Chem. 46:625-30, 2000; U.S. Pat. No. 6,140,055). Several of such methods are also currently available for performing real-time TMA (News release, Tosoh, Gen-Probe Sign Cross-Licensing Agreement For Nucleic Acid Testing Technologies, Dec. 9, 2003). Again, since these technology use target-specific probes for detection, optimization of each probe for each target is usually required. This is a time-consuming step and requires specific software and/or experience.

Currently available labeled primers can have a secondary structure that is complex and in some instances must be synthesized using specialized procedures. For example LUX™ primers (Invitrogen Corp.) are fluorescently labeled on the 3′-end and have a stem-loop structure that must be denatured for the primer to work efficiently. The design and optimization of the LUX™ primer is also a time-consuming step, which requires specific software.

Several publications described probes that contain only one fluorophore for use in detecting the presence of a particular nucleic acid (for example, see U.S. Pat. No. 6,699,661; U.S. Pat. No. 6,495,326; and U.S. Pat. No. 6,492,121 (all to Kurane et al.); U.S. Pat. No. 6,635,427 (Wittwer et al.); Kurata et al., Nucl. Acids Res. 29:e34, 2001; Torimura et al., Analyt. Sci, 17:155-60, 2001; and Crockett et al., Analyt. Biochem. 290:89-97, 2001). In these examples, the fluorophore is present on the very end of the probe and the fluorescent signal is either enhanced or quenched in the presence of the target nucleic acid sequence, depending on the particular design of the probe. In most cases, the labeled primer specifically hybridizes to the target nucleic acid sequence.

Guo and Milewicz (Biotech. Lett. 25:2079-83, 2003) described universal fluorescent tag primers labeled on the 5′-end that are not sequence specific. The labeled fluorescent tag universal primer, in combination with two sequence-specific primers, are use to amplify a target nucleic acid sequence.

Yamane (Nucl. Acids Res. 30:e97, 2002) described a MagniProbe that has an internal fluorophore and an internal intercalator. The fluorescence is quenched by the intercalator in the absence of a target sequence. Upon hybridization with the target sequence, the probe emits fluorescence due to the interference in quenching by intercalation.

Nazarenko et al. (Nucl. Acids Res. 30:e37, 2002) described a probe with a single fluorophore near the 3′-end (but no quencher), and addition of 5-7 base pairs to the 5′-end of the sequence-specific probe, wherein the signal from the fluorophore is increased in the presence of the target sequence.

Narayanan et al (U.S. Pat. Appl. Pub. No. 20060188902) described a universal primer, which includes labeled nucleotide flanked on both sides a nucleotide whose complement nucleotides changes a detectable signal from the label when the universal primer hybridizes with its complementary nucleic acid molecule.

All of the above technology uses target-specific sequences for probe design. Design and optimization of each probe for each target is required. This is a costly, time-consuming and often challenging and difficult process. When the useful sequences of a particular target is limited, it is difficult to design sequence specific probes and primers.

In contrast, the present invention describes a signal primer and a detection means design to overcome difficulties described above.

### 3. SUMMARY OF THE INVENTION

The application relates to kits and compositions comprising a signal primer that comprises (i) a first target-specific (TS1) sequence which hybridizes specifically to a nucleic acid target, and (ii) a signal (Sg) sequence upstream of the TS1 sequence; and a detection means for detecting the presence of the complementary sequence to the signal sequence, as well as methods of using the signal primer and detection means to amplify and detect one or more nucleic acid targets.

One aspect of the invention relates to a kit for selective amplification and detection of at least one nucleic acid target. In one embodiment, the kit comprises one or more container means that comprises:
(a) a signal primer that comprises
   (i) a first target-specific (TS1) sequence which hybridizes specifically to nucleic acid target, and
   (ii) a signal (Sg) sequence upstream of the TS1 sequence; and
(b) a capture moiety that comprises a solid support linked with a second target-specific (TS2) sequence which hybridizes specifically to the nucleic acid target, wherein the TS1 and TS2 sequences hybridize to non-overlapping regions of the nucleic acid target; and
(c) an opposite primer that comprises a third target-specific (TS3) sequence selected from a region of the nucleic acid target that is upstream of the region to which the TS1 sequence hybridizes; and
(d) a detection means for detecting the presence of the complementary sequence to the signal sequence.

Optionally, the kit further comprises a block oligonucleotide.

In certain embodiments, the signal sequence is not found in the nucleic acid target or its complementary sequence.

In a specific embodiment, the nucleic acid target is a positive-sense (or plus (+)-strand), and the TS1 and TS2 sequences are negative-sense (or minus (−)-strands) and the TS3 sequence is positive-sense (+). In another specific embodiment, the nucleic acid target is negative-sense (−), and the TS1 and TS2 sequences are positive-sense (+) and the TS3 sequence is negative-sense (−).

In certain embodiments, the opposite primer in the kit further comprises a promoter (Pm) sequence upstream of the TS3 sequence.

The kits of the invention are useful for detecting multiple nucleic acid targets. In these situations, the kit comprises, for each different nucleic acid target, a different set of signal primer, capture moiety, and opposite primer.

In some embodiments, the kit comprises more than one signal primer for each nucleic acid target. In some embodiments, the kit comprises more than one capture moiety for each nucleic acid target. In some embodiments, the kit comprises more than one opposite primer for each nucleic acid target. In some embodiments, the kit comprises one or more signal primer and/or one or more capture moiety and/or one or more opposite primer for each nucleic acid target.

In certain embodiments, the kit further comprises a TAG primer which comprises a TAG sequence, and the signal primer further comprises the TAG sequence upstream of the signal sequence. In one embodiment, the TAG primer does not comprise the TS1 sequence or the signal sequence. In another embodiment, the TAG sequence is not found in the signal sequence, the nucleic acid target, or their respective complementary sequences. In yet another embodiment, the TAG primer does not comprise the TS1 sequence or the signal sequence and is not found in the signal sequence, the nucleic acid target, or their respective complementary sequences.

In certain embodiments, the kit further comprises a first TAG (TAG1) primer which comprises a first TAG (TAG1) sequence and a second TAG (TAG2) primer which comprises a second TAG (TAG2) sequence, and the signal primer further comprises the TAG1 sequence upstream of the signal sequence and the opposite primer further comprises the TAG2 sequence upstream of the TS3 sequence. In one embodiment, the signal primer and the opposite primer are connected together at their respective 5′-end by a connector such as an oligoethylene glycol bridge. In those embodiments in which the opposition primer further comprises the TAG2 sequence and a promoter (Pm) sequence upstream of the TS3 sequence, the TAG2 sequence is downstream of the promoter sequence and upstream of the TS3 sequence.

In one embodiment, neither one of the TAG1 and TAG2 primers comprise the TS1 sequence or the signal sequence. In another embodiment, neither one of the TAG1 and TAG2 sequences is found in the signal sequence, the nucleic acid target, or their respective complementary sequences. In yet another embodiment, neither one of the TAG1 and TAG2 primers comprise the TS1 sequence or the signal sequence and is found in the signal sequence, the nucleic acid target, or their respective complementary sequences. Preferably, the TAG1 and TAG2 sequences are different and not complementary to each other. Preferably, the TAG1 and TAG2 sequences do not hybridize to each other.

In certain embodiments, each of the signal primer, the capture moiety, the opposite primer and the detection means in the kit is placed in separate container means.

In certain embodiments, the signal primer and the capture moiety are placed in a first container means, and the opposite primer and the detection means in the kit are placed in one or two container means different from the first container means.

In certain embodiments, the capture moiety in the kit comprises (i) the solid support which is attached with a linker oligonucleotide, and (ii) a capture oligonucleotide comprising the TS2 sequence, and, downstream from the TS2 sequence, the complementary sequence to the linker oligonucleotide. In some embodiments, the linker oligonucleotide comprises a poly(B) tail, wherein B is a nucleoside. Nucleosides are glycosylamines consisting of a nucleobase (or base) bound to a ribose or deoxyribose sugar. Examples of nucleosides include adenosine (A), guanosine (G), cytidine (C), uridine (U), and thymidine (T).

In certain embodiments, the solid support of the capture moiety in the kit is selected from the group consisting of nitrocellulose, nylon, glass, polyacrylate, mixed polymers, polystyrene, silane polypropylene, magnetic particle, microtiter plate, column, fiber, and capillary.

In certain embodiments, the detection means in the kit comprises the signal sequence. In some embodiments, the detection means is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

Optionally, the kits of the invention further comprises an instruction manual describing, for example, the component(s) within each container means, the order of using the one or more container means, etc.

Another aspect of the invention relates to a composition comprising the signal primer and the detection means described above. In one embodiment, the signal primer comprises (i) a first target-specific (TS1) sequence which hybridizes specifically to a nucleic acid target, and (ii) a signal (Sg) sequence upstream of the TS1 sequence, wherein the signal sequence is preferably not found in the nucleic acid target or its complementary sequence, and the detection means is for detecting the presence of the complementary sequence to the signal sequence.

In certain embodiments, the signal primer further comprises, upstream of the signal sequence, a TAG sequence which is preferably not found in the signal sequence, the nucleic acid target, or their respective complementary sequences.

In certain embodiments, the detection means comprises the signal sequence, and preferably, is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

In certain embodiments, the composition further comprises a single-stranded RNA comprising, in the direction from the 5′-end to the 3′-end, a region of the nucleic acid target, a complementary sequence to the TS1 sequence, and a complementary sequence to the signal sequence (the Sg′ sequence). Preferably, this composition is an aqueous solution wherein the detection means is hybridized to the Sg′ sequence of the single-stranded RNA.

In certain embodiments, the composition further comprises a double-stranded DNA comprising, in the direction from the 5′-end to the 3′-end, a region of the nucleic acid target, a complementary sequence to the TS1 sequence, and a complementary sequence to the signal sequence (the Sg′ sequence). Preferably, this composition is an aqueous solution wherein the detection means is hybridized to the Sg′ sequence of the double-stranded DNA.

Another aspect of the invention relates to a method for selective amplification and detection of at least one nucleic acid target, using, for example, a transcription-mediated amplification (TMA) reaction. In one embodiment, the method comprises the steps of:
(a) mixing a sample comprising or is suspected of comprising a nucleic acid target with a signal primer and a capture moiety, wherein the signal primer comprises
   (i) a first target-specific (TS1) sequence which hybridizes specifically to the nucleic acid target, and
   (ii) a signal (Sg) sequence upstream of the TS1 sequence, wherein the signal sequence is preferably not found in the nucleic acid target or its complementary sequence, and
   (iii) a TAG sequence upstream of the signal sequence, wherein the TAG sequence is preferably not found in the signal sequence, the nucleic acid target, or their respective complementary sequences and which preferably does not comprise the TS1 sequence or the signal sequence; and
the capture moiety comprises a solid support linked with a second target-specific (TS2) sequence which hybridizes specifically to the nucleic acid target, wherein the TS1 and TS2 sequences hybridize to non-overlapping regions of the nucleic acid target;
wherein said mixing is conducted under a condition for the TS1 and TS2 sequences to hybridize to the nucleic acid target and form a complex comprising the nucleic acid target hybridized with the signal primer and the capture moiety;
(b) isolating or purifying the complex to separate it from excess signal primer not hybridized to the nucleic acid target;
(c) mixing the isolated or purified complex with an opposite primer and a TAG primer in a mixture, wherein the TAG primer comprises the TAG sequence, and the opposite primer comprises (i) a third target-specific (TS3) sequence selected from a region of the nucleic acid target that is upstream of the region to which the TS1 sequence hybridizes, and (ii) a promoter (Pm) sequence upstream of the TS3 sequence;
(d) amplifying the nucleic acid target present in Step (c) to generate a plurality of an amplified RNA product that comprises, in the direction from the 5′-end to the 3′-end, the TS3 sequence, the complementary sequence to the TS1 sequence, the complementary sequence to the signal sequence, and the complementary sequence to the TAG sequence; and
(e) detecting the presence of the amplified RNA product with a detection means which is capable of detecting the presence of the complementary sequence to the signal sequence, wherein the detection means is introduced into the mixture during or after Step (c).

Another aspect of the invention relates to a method for selective amplification and detection of at least one nucleic acid target (RNA or DNA), using, for example, a polymerase chain reaction (PCR). In one embodiment, the method comprises the steps of:
(a) mixing a sample comprising or is suspected of comprising a nucleic acid target with a signal primer and a capture moiety, wherein the signal primer comprises
   (i) a first target-specific (TS1) sequence which hybridizes specifically to the nucleic acid target, and
   (ii) a signal (Sg) sequence upstream of the TS1 sequence, wherein the signal sequence is preferably not found in the nucleic acid target or its complementary sequence, and
   (iii) a TAG sequence upstream of the signal sequence, wherein the TAG sequence is preferably not found in the signal sequence, the nucleic acid target, or their respective complementary sequences and which preferably does not comprise the TS1 sequence or the signal sequence; and
the capture moiety comprises a solid support linked with a second target-specific (TS2) sequence which hybridizes specifically to the nucleic acid target, wherein the TS1 and TS2 sequences hybridize to non-overlapping regions of the nucleic acid target;
wherein said mixing is conducted under a condition for the TS1 and TS2 sequences to hybridize to the nucleic acid target and form a complex comprising the nucleic acid target hybridized with the signal primer and the capture moiety;
(b) isolating or purifying the complex to separate it from excess signal primer not hybridized to the nucleic acid target;
(c) mixing the isolated or purified complex with an opposite primer and a TAG primer in a mixture, wherein the TAG primer comprises the TAG sequence, and the opposite primer comprises (i) a third target-specific (TS3) sequence selected from a region of the nucleic acid target that is upstream of the region to which the TS1 sequence hybridizes, and optionally (e.g., when the nucleic acid target is RNA) (ii) a promoter (Pm) sequence upstream of the TS3 sequence;
(d) amplifying the nucleic acid target present in Step (c) to generate a plurality of an amplified DNA product that comprises a strand, in the direction from the 5′-end to the 3′-end, the TS3 sequence, the complementary sequence to the TS1 sequence, the complementary sequence to the signal sequence, and the complementary sequence to the TAG sequence; and
(e) detecting the presence of the amplified DNA product with a detection means which is capable of detecting the presence of the complementary sequence to the signal sequence, wherein the detection means is introduced into the mixture during or after Step (c).

In certain embodiments, the method uses for each different nucleic acid target, a different set of signal primer, capture moiety, and opposite primer. In a specific embodiment, the method is for selective amplification and detection of two or more different nucleic acid targets using a TMA reaction. In one embodiment, the method comprises the steps of:
(a) mixing a sample comprising or is suspected of comprising at least a first nucleic acid target (Ta) and a second nucleic acid target (Tb) with a Ta-specific signal primer, a Ta-specific capture moiety, a Tb-specific signal primer and a Tb-specific capture moiety, wherein the Ta-specific signal primer comprises
   (i) a first Ta-specific (TS1a) sequence which hybridizes specifically to the Ta nucleic acid target, and
   (ii) a signal (Sg) sequence upstream of the TS1a sequence, wherein the signal sequence is preferably not found in the Ta nucleic acid target, the Tb nucleic acid target, or their respective complementary sequences, and
   (iii) a TAG sequence upstream of the signal sequence, wherein the TAG sequence is preferably not found in the signal sequence, the Ta nucleic acid target, the Tb nucleic acid target, or their respective complementary sequences, and which preferably does not comprise the TS1 sequence or the signal sequence, and
the Ta-specific capture moiety comprises a solid support linked with a second Ta-specific (TS2a) sequence which hybridizes specifically to the Ta nucleic acid target, wherein the TS1a and TS2a sequences hybridize to non-overlapping regions of the Ta nucleic acid target, and the Tb-specific signal primer comprises
   (i) a first Tb-specific (TS1b) sequence which hybridizes specifically to the Tb nucleic acid target, and
   (ii) the signal sequence upstream of the TS1b sequence, and
   (iii) the TAG sequence upstream of the signal sequence, and the Tb-specific capture moiety comprises a solid support linked with a second Tb-specific (TS2b) sequence which hybridizes specifically to the Tb nucleic acid target, wherein the TS1b and TS2ba sequences hybridize to non-overlapping regions of the Tb nucleic acid target;
wherein said mixing is conducted under a condition for the TS1a and TS2a sequences to hybridize to the Ta nucleic acid target and form a Ta complex comprising the Ta nucleic acid target hybridized with the Ta-specific signal primer and the Ta-specific capture moiety and for the TS1b and TS2b sequences to hybridize to the Tb nucleic acid target and form a Tb complex comprising the Tb nucleic acid target hybridized with the Tb-specific signal primer and the Tb-specific capture moiety;
(b) isolating or purifying the Ta and Tb complexes to separate them from excess Ta-specific signal primer not hybridized to the Ta nucleic acid target and from excess Tb-specific signal primer not hybridized to the Tb nucleic acid target;
(c) mixing the isolated or purified Ta and Tb complexes with a Ta-specific opposite primer, a Tb-specific opposite primer and a TAG primer in a mixture, wherein the TAG primer comprises the TAG sequence, the Ta-specific opposite primer comprises
   (i) a third Ta-specific (TS3a) sequence selected from a region of the Ta nucleic acid target that is upstream of the region to which the TS1a sequence hybridizes, and
   (ii) a first promoter (Pma) sequence upstream of the TS3a sequence, and the Tb-specific opposite primer comprises
   (i) a third Tb-specific (TS3b) sequence selected from a region of the Tb nucleic acid target that is upstream of the region to which the TS1b sequence hybridizes, and
   (ii) a second promoter (Pmb) sequence upstream of the TS3b sequence;
(d) amplifying the Ta and Tb nucleic acid targets present in Step (c) to generate a plurality of an amplified Ta RNA product and an amplified Tb RNA product, wherein the amplified Ta RNA product comprises, in the direction from the 5′-end to the 3′-end, the TS3a sequence, the complementary sequence to the TS1a sequence, the complementary sequence to the signal sequence, and the complementary sequence to the TAG sequence, and the amplified Tb RNA product comprises, in the direction from the 5′-end to the 3′-end, the TS3b sequence, the complementary sequence to the TS1b sequence, the complementary sequence to the signal sequence, and the complementary sequence to the TAG sequence; and
(e) detecting the presence of the amplified Ta and Tb RNA products with a detection means which is capable of detecting the presence of the complementary sequence to the signal sequence, wherein the detection means is introduced into the mixture during or after Step (c).

In another specific embodiment, the method is for selective amplification and detection of two or more different nucleic acid targets (RNA or DNA) using PCR. In one embodiment, the method comprises the steps of:
(a) mixing a sample comprising or is suspected of comprising at least a first nucleic acid target (Ta) and a second nucleic acid target (Tb) with a Ta-specific signal primer, a Ta-specific capture moiety, a Tb-specific signal primer and a Tb-specific capture moiety, wherein the Ta-specific signal primer comprises
   (i) a first Ta-specific (TS1a) sequence which hybridizes specifically to the Ta nucleic acid target, and
   (ii) a signal (Sg) sequence upstream of the TS1a sequence, wherein the signal sequence is preferably not found in the Ta nucleic acid target, the Tb nucleic acid target, or their respective complementary sequences, and
   (iii) a TAG sequence upstream of the signal sequence, wherein the TAG sequence is preferably not found in the signal sequence, the Ta nucleic acid target, the Tb nucleic acid target, or their respective complementary sequences, and which preferably does not comprise the TS1 sequence or the signal sequence, and
the Ta-specific capture moiety comprises a solid support linked with a second Ta-specific (TS2a) sequence which hybridizes specifically to the Ta nucleic acid target, wherein the TS1a and TS2a sequences hybridize to non-overlapping regions of the Ta nucleic acid target, and
   (i) the Tb-specific signal primer comprises
   (i) a first Tb-specific (TS1b) sequence which hybridizes specifically to the Tb nucleic acid target, and
   (ii) the signal sequence upstream of the TS1b sequence, and
   (iii) the TAG sequence upstream of the signal sequence, and the Tb-specific capture moiety comprises a solid support linked with a second Tb-specific (TS2b) sequence which hybridizes specifically to the Tb nucleic acid target, wherein the TS1b and TS2ba sequences hybridize to non-overlapping regions of the Tb nucleic acid target;
wherein said mixing is conducted under a condition for the TS1a and TS2a sequences to hybridize to the Ta nucleic acid target and form a Ta complex comprising the Ta nucleic acid target hybridized with the Ta-specific signal primer and the Ta-specific capture moiety and for the TS1b and TS2b sequences to hybridize to the Tb nucleic acid target and form a Tb complex comprising the Tb nucleic acid target hybridized with the Tb-specific signal primer and the Tb-specific capture moiety;
(b) isolating or purifying the Ta and Tb complexes to separate them from excess Ta-specific signal primer not hybridized to the Ta nucleic acid target and from excess Tb-specific signal primer not hybridized to the Tb nucleic acid target;
(c) mixing the isolated or purified Ta and Tb complexes with a Ta-specific opposite primer, a Tb-specific opposite primer and a TAG primer in a mixture, wherein the TAG primer comprises the TAG sequence, the Ta-specific opposite primer comprises
   (i) a third Ta-specific (TS3a) sequence selected from a region of the Ta nucleic acid target that is upstream of the region to which the TS1a sequence hybridizes, and optionally (e.g., when the Ta nucleic acid target is RNA)
   (ii) a first promoter (Pma) sequence upstream of the TS3a sequence, and the Tb-specific opposite primer comprises
   (i) a third Tb-specific (TS3b) sequence selected from a region of the Tb nucleic acid target that is upstream of the region to which the TS1b sequence hybridizes, and optionally (e.g., when the Ta nucleic acid target is RNA)
   (ii) a second promoter (Pmb) sequence upstream of the TS3b sequence;
(d) amplifying the Ta and Tb nucleic acid targets present in Step (c) to generate a plurality of an amplified Ta DNA product and an amplified Tb DNA product, wherein the amplified Ta DNA product comprises a strand, in the direction from the 5′-end to the 3′-end, the TS3a sequence, the complementary sequence to the TS1a sequence, the complementary sequence to the signal sequence, and the complementary sequence to the TAG sequence, and the amplified Tb DNA product comprises a strand, in the direction from the 5′-end to the 3′-end, the TS3b sequence, the complementary sequence to the TS1b sequence, the complementary sequence to the signal sequence, and the complementary sequence to the TAG sequence; and
(e) detecting the presence of the amplified Ta and Tb DNA products with a detection means which is capable of detecting the presence of the complementary sequence to the signal sequence, wherein the detection means is introduced into the mixture during or after Step (c).

In certain embodiments, the capture moiety comprises (i) the solid support which is attached with a linker oligonucleotide, and (ii) a capture oligonucleotide comprising the TS2 sequence, and, downstream from the TS2 sequence, the complementary sequence to the linker oligonucleotide.

In certain embodiments, the linker oligonucleotide comprises a poly(B) tail, wherein B is a nucleoside, such as adenosine (A), guanosine (G), cytidine (C), uridine (U), or thymidine (T).

In certain embodiments, the solid support is selected from the group consisting of nitrocellulose, nylon, glass, polyacrylate, mixed polymers, polystyrene, silane polypropylene, magnetic particle, microtiter plate, column, fiber, and capillary.

In certain embodiments, the detection means comprises the signal sequence, and preferably, is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

Optionally, the method comprises mixing in Step (c) a block oligonucleotide that restricts primer extension during the amplificaiton process.

### 3.1 ABBREVIATIONS AND TERMS

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

As used herein, the term “3′-end” means the end of a nucleic acid sequence where the 3′ position of the terminal residue is not bound by a nucleotide.

As used herein, the term “5′-end” means the end of a nucleic acid sequence where the 5′ position of the terminal residue is not bound by a nucleotide.

As used herein, the phrase “amplifying [a nucleic acid molecule]” means to increase the number of copies of a nucleic acid molecule. The resulting amplification products are called “amplicons.”

As used herein, the term “change” means to become different in some way, for example to be altered, such as increased or decreased. A detectable change is one that can be detected, such as a change in the intensity, frequency or presence of a signal, such as fluorescence. In particular examples, the detectable change is a reduction in fluorescence intensity.

As used herein, the term “complementary” means binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one single-stranded (ss) DNA molecule can bond to 3′-TAGC-5′ of another ssDNA to form a double-stranded (ds) DNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

As used herein, the term “fluorophore” means a chemical compound, which when excited by exposure to a particular wavelength of light, emits light (fluoresces), for example at a different wavelength of light. Exemplary fluorophores include, but are not limited to: 6-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC.RTM. (Applied Biosystems), LC Red 640, LC Red 705, Yakima yellow, as well as derivatives thereof. Also encompassed by the term “fluorophore” are luminescent molecules, which are chemical compounds which do not require exposure to a particular wavelength of light to fluoresce; luminescent compounds naturally fluoresce. Therefore, the use of luminescent signals can eliminate the need for an external source of electromagnetic radiation, such as a laser.

As used herein, the phrase “hybridization [of a nucleic acid]” means when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acids used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). Tm is the temperature at which 50% of a given strand of nucleic acid is hybridized to its complementary strand.

As used herein, the term “stringent conditions” encompasses conditions under which hybridization only will occur if there is less than 25% mismatch between the hybridization molecule and the target sequence. Conditions of “moderate stringency are those under which molecules with more than 25% sequence mismatch will not hybridize; conditions of “medium stringency” are those under which molecules with more than 1.5% mismatch will not hybridize; conditions of “high stringency” are those under which sequences with more than 10% mismatch will not hybridize; and conditions of “very high stringency” are those under which sequences with more than 5% mismatch will not hybridize.

Moderately stringent hybridization conditions include those under which hybridization is performed at, for example, about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions include those under which hybridization is performed at, for example, about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

The complementary nucleic acid sequences described herein can hybridize under stringent, moderately stringent, and/or highly stringent condition.

As used herein, the term “isolated [biological component]” means a biological component (such as a nucleic acid molecule) which has been substantially separated, produced apart from, or purified away from other biological components. Nucleic acid molecules which have been “isolated” include nucleic acid molecules purified by standard purification methods, as well as those chemically synthesized. Isolated does not require absolute purity, and can include nucleic acid molecules that are at least 50% isolated, such as at least 75%, 80%, 90%, 95%, 98%, 99% or even 100% isolated.

As used herein, the term “label” means an agent capable of detection, for example by spectrophotometry, flow cytometry, or microscopy. For example, a label can be attached to a nucleotide, thereby permitting detection of the nucleotide, such as detection of the nucleic acid molecule of which the nucleotide is a part of Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

As used herein, the term “nucleic acid molecule” means a deoxyribonucleotide or ribonucleotide polymer, which can include analogues of natural nucleotides that hybridize to nucleic acid molecules in a manner similar to naturally occurring nucleotides. In a particular example, a nucleic acid molecule is a ssDNA or RNA molecule, such as a primer. In another particular example, a nucleic acid molecule is a ds DNA, such as a target nucleic acid. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 51-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine.

As used herein, the term “primer” means an oligonucleotide, which, when hybridized to its complementary nucleic acid targets, allows strand extension by a polymerase. Primer pairs bracketing an amplicon can be used for amplification of a nucleic acid sequence, for example by PCR, TMA or other nucleic-acid amplification methods. In preferred embodiments, the signal primer of the invention comprising the signal sequence and the TS1 sequence but not the TAG sequence is no more than 40, 50, 60, 70 or 80, preferably no more than 60, bases in length. In preferred embodiments, the signal primer containing the TAG sequence, the signal sequence, and the TS1 sequence is no more than 60, 70, 80, 90 or 100, preferably no more than 80, bases in length. In preferred embodiments, the opposite primer containing a promoter sequence and the TS3 sequence is no more than 45, 55, 65 or 75, preferably no more than 65, bases in length.

As used herein, the phrase “quantitating a nucleic acid molecule” means determining or measuring a quantity (such as a relative quantity) of nucleic acid molecule present, such as the number of amplicons or the number of nucleic acid molecules present in a sample. In particular examples, it is determining the relative amount or actual number of nucleic acid molecules present in a sample.

As used herein, the term “quenching of fluorescence” means a reduction of fluorescence. For example, quenching of a fluorophore's fluorescence on a sequence occurs when a quencher molecule (such as guanosine) is present in sufficient proximity to the fluorophore that it reduces the fluorescence signal of the reporter molecule during complementary strand synthesis.

As used herein, the term “real-time PCR” means a method for detecting and measuring products generated during each cycle of a PCR, which are proportionate to the amount of nucleic acid target prior to the start of PCR. The information obtained, such as an amplification curve, can be used to quantitate the initial amounts of nucleic acid target.

As used herein, the term “real-time TMA” means a method for detecting and measuring products generated during the process of TMA, which are proportionate to the amount of nucleic acid target prior to the start of TMA. The information obtained, such as an amplification curve, can be used to quantitate the initial amounts of nucleic acid target.

As used herein, the term “recombinant nucleic acid molecule” is one that has a sequence that is not naturally occurring or has a sequence that is made by an artificial combination of two otherwise separated segments of sequence. This artificial combination can be accomplished, for example, by chemical synthesis or by the artificial manipulation of isolated segments of nucleic acid molecules, for example, by genetic engineering techniques.

As used herein, the term “sample” means biological samples such as samples containing nucleic acid molecules, such as genomic DNA, cDNA, RNA, mRNA, rRNA, or combinations thereof. Samples can be obtained from the cells of a subject, such as those present in peripheral blood, urine, saliva, tissue biopsy, surgical specimen, fine needle aspirates, amniocentesis samples and autopsy material.

As used herein, the term “target-specific sequence” means a nucleic acid sequence that can substantially hybridize with a nucleic acid target, such as under a stringent, moderately stringent, or highly stringent condition. In particular examples, such target-specific sequences are at least nine, ten, fifteen, twenty, thirty or more nucleotides long. Preferably, the target-specific sequences are about twenty nucleotides long.

As used herein, the term “signal” means an indicator, such as a detectable physical quantity from which information can be obtained. In one example, a label emits a signal capable of detection, such as a fluorescent signal.

As used herein, the term “subject” means living multi-cellular vertebrate organisms, including human and veterinary subjects, such as cows, pigs, horses, dogs, cats, birds, reptiles, and fish.

As used herein, the term “synthesis of a nucleic acid molecule” means building up a molecule from its component parts, for example by replicating a nucleic acid molecule. Examples include, but are not limited to, DNA synthesis and RNA-dependent DNA synthesis using reverse transcriptase.

As used herein, the term “nucleic acid target” means a nucleic acid molecule whose detection, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule in a cell (which can include host RNAs (such as mRNA) and DNAs (such as genomic or cDNA), as well as other nucleic acid molecules such as viral, bacterial or fungal nucleic acid molecules), the amplification of which is intended. Purification or isolation of the target nucleic acid molecule, if needed, can be conducted by methods known to those in the art, such as by using a commercially available purification kit or the like.

As used herein, the term “signal sequence” means any sequence that works well in the present invention. The signal sequences can be contained or not contained in the nucleic acid target. In a single nucleic acid target amplification and detection assay, the signal sequence is preferably not found in the nucleic acid target. In a multiple nucleic acid target amplification and detection assay, the signal sequence can be from a region of one of the nucleic acid target. The signal sequences can be at any position in the signal primer or detection means, as long as it generates signal change when it is hybridized with its complementary sequence. The change in the detectable signal from the label upon hybridization of the detection probe with its complementary sequence can be an increase or a decrease in the detectable signal, such as an increase or decrease of at least 10%, such as at least 20%, at least 50%, at least 75%, or at least 90%, as compared to a control, such as an amount of signal when the universal tag is not hybridized to its complementary sequence (for example when it is unbound in solution). In examples in which the label is a fluorescent label, the intensity of the fluorescence emitted by the label changes in a predictable way (for example by decreasing or dissipating when the universal sequence hybridizes to its complementary sequence). In particular examples, the change in signal is a decrease in fluorescence, such as a quenching of fluorescence. For example, the fluorescence can decrease by at least 10%, such as at least 20%, at least 50%, at least 75%, or at least 90%, when the signal sequence is hybridized to its complementary sequence, as compared to a control, such as an amount of fluorescence when the universal tag is not hybridized to its complementary sequence.

The described signal sequences which contained in primers or detection probes can be any length that permits detection of a change in signal from the label on the detection probe, when the detection probe hybridizes with its complementary sequence. In particular examples, the signal sequence is at least 8 nucleotides, at least 12 nucleotides, at least 15 nucleotides, at least 18 nucleotides, at least 21 nucleotides, at least 25 nucleotides, at least 30 nucleotides, at least 30 nucleotides such as 8-25 nucleotides, 12-25 nucleotides, 15-25 nucleotides, 18-25 nucleotides, 21-25 nucleotides, 8-15 nucleotides, 12-15 nucleotides, or 15-15 nucleotides, etc.

As used herein, the term “upstream” and “downstream” refer to a relative position in DNA or RNA. Each strand of DNA or RNA has a 5′-end and a 3′-end, so named for the carbons on the deoxyribose ring. Relative to the position on the strand, downstream is the region towards the 3′-end of the strand, and upstream is the region towards the 5′-end of the strand. Since DNA strands run in opposite directions, downstream on one strand is upstream on the other strand.

### 4. BRIEF DESCRIPTION OF THE FIGURES

FIG. 1. Single target amplification and detection using a capture moiety, a signal primer, a TAG primer, and a detection means of the invention.

FIG. 2. Multiple target amplification and detection using target-specific capture moieties, target-specific signal primers with a single TAG sequence, a TAG primer, and a detection means of the invention.

FIG. 3. Multiple target amplification and detection using target-specific capture moieties, target-specific signal primers with dual TAG sequences, a non-promoter TAG primer, a promoter TAG primer, and a detection means of the invention.

FIG. 4. Raw curve of amplification in which no target or 10 U. urealyticum cells were spiked into the target capture reagent or amplification reagent.

FIG. 5. Raw curve of amplification when different number of E. coli cells were added into the target capture reagent.

FIG. 6. Raw curve of amplification when either 100 or 0 E. coli cells were added into the target capture reagent using a method of the invention (FIG. 6A) or a method of an existing technology (FIG. 6B).

FIG. 7. Raw curve of amplification when 10⁶ copies of HCV (FIG. 7A), 10⁶ copies of HBV (FIG. 7B), 10⁶ copies of HIV (FIG. 7C), and 10⁴ copies each of HCV, HBV and HIV (FIG. 7D) in vitro transcripts were added into the target capture reagent.

### 5. DETAILED DESCRIPTION OF THE INVENTION

Section 5.1 below describes in detail the signal primer and detection means (e.g., detection probe) in the compositions and kits of the invention. Section 5.2 below describes in detail methods of using the signal primer and detection means of the invention.

### 5.1 SIGNAL PRIMER AND DETECTION MEANS OF THE INVENTION

Conventional technology uses target-specific sequences for probe design. Design and optimization of each probe for each target is required. This is a costly, time-consuming and often challenging and difficult process. When the useful sequences of a particular target is limited, it is difficult to design sequence specific probes and primers.

In contrast, the current invention adds a signal sequence upstream of the target-specific sequence to construct a “signal primer”. This design of “signal primer” and its corresponding detection probe, as described in the present invention, significantly facilitates the signal primer and detection probe design to overcome difficulties described above. In addition, once one or more working “signal sequences” is identified, it can be used to detect different targets in different systems. Therefore, this invention describes a method that can be used in many nucleic acid detection technologies (including but not limited to PCR, NASBA and TMA) by using a signal primer comprising a signal sequence combined with a detection means comprising the signal sequence, instead of requiring target-specific detection probes for each target nucleic acid molecule. The requirement and expense of designing and optimizing target-specific detection probes and primers for each target nucleic acid molecule or variant in various nucleic acid detection technologies is no longer needed.

The present invention also provides a very simple system for multiplex nucleic acid detection where target-specificity is not strictly required, such as in multiplex sexually transmitted diseases detection and blood bank screen for viruses. For example, a single detection probe containing a signal sequence (see Example 6.4, below), can be used in PCR or TMA blood bank multiplex screen of HIV, HCV, and HBV, In this example, the signal sequence can be contained in the signal primer for each target, while only one labeled detection probe containing the signal sequence, is needed.

The present application also relates to the use of signal sequences in assessing the progress of a reaction, such as real-time PCR, real-time TMA or real-time NASBA, or for assessing the progress of melting duplex DNA, such as an amplicon.

The signal from the label (i.e., detection means) changes when the labeled nucleic acid sequence is hybridized to its complementary sequence. The change in the signal can be an increase or a decrease. The resulting change in detectable signal is proportional to the amount of amplicon produced and therefore occurs only when a complementary strand is synthesized. The signal can be detected by a variety of devices, such as fluorescent microtiter plate readers, spectrofluorometers, fluorescent imaging systems, real-time PCR machines, and chemiluminacent measurement instruments.

The current invention can be used in many currently available nucleic acid amplification and detection technologies, for example molecular beacons (e.g., Scorpion probes), TaqMan probes, fluorescence resonance energy transfer (FRET) probes, induced FRET (iFRET) probes, minor grove binder (MGB) probes (e.g., MGB Eclipse probes), molecular torches, hybridization switch probes, etc.

The described method can be used to attach a signal sequence to a target-specific sequence without significantly affecting the sensitivity and specificity of the amplification reaction. Ideally, the target-specific sequence of the signal primer specifically recognizes a target nucleic acid molecule. Preferably, the signal primer hybridizes to a target nucleic acid molecule under moderately stringent or highly stringent hybridization conditions.

A signal sequence can be hybridized to the target nucleic acid molecule of interest, thereby generating a sequence complementary to the signal sequence that can hybridize to a labeled detection probe also containing the signal sequence. Upon hybridization between the labeled detection probe and the complementary sequence of the signal sequence, a signal change is generated. The signal change can be detected or quantified to detect or quantify the amount of target nucleic acid molecule present in a sample.

Also provided by the present disclosure are kits that include one or more signal primers and detection probes each containing one or more signal sequences. The kits can further include a ligase to permit joining of the 3′-end of a signal sequence to a 5′-end of a sequence-specific forward or reverse primer. In some examples, the kit also includes one or more sequence-specific forward or reverse primers, such as primers that recognize and can used to amplify a target sequence of interest.

Arrays, such as a DNA microarray, can benefit from the use of the signal primer and detection probe of the invention (both containing a signal sequence). When amplified target nucleic acid molecules containing the complementary sequence of the signal sequence as described by this invention are captured at specific spots of a microarray, the described detection probe containing the signal sequence can be hybridized to such amplified nucleic acid and provide signal (e.g., fluorescence) at the spots.

The described signal primer/detection probe combination provides an approach to detect, and in some examples quantify, a target nucleic acid molecule. Use of the signal sequence is shown herein to provide a highly sensitive detection method, which permits detection of small quantities of a target nucleic acid molecule, such as DNA or RNA.

For example, the present disclosure provides methods of detecting a target nuclei' acid molecule. The method includes incubating a sample containing target nucleic acid molecules (such as DNA or RNA) with a signal sequence which is linked to a target-specific sequence (i.e., signal primer), and with the corresponding opposite primer not containing the signal sequence, and labeled detection probe also containing the same signal sequence. The sample, labeled detection probe containing the signal sequence, signal primer containing the signal sequence, and opposite primer not containing the signal sequence, are incubated under conditions sufficient to permit amplification of the target nucleic acid molecule. A change in signal from the label on the detection probe is monitored, wherein a change in signal indicates the presence of the target nucleic acid molecule. In particular examples, both the signal primer and the opposite primer contain signal sequences.

In some examples, the change in signal is monitored during the amplification reaction, for example in real-time as the amplicons are formed. In other or additional examples, the change in signal is monitored after the amplification, for example by exposing the resulting amplicons to increased temperature to generate a melting curve. Melting curve analysis can be used to confirm the presence of a target nucleic acid molecule, and can also be used to distinguish polymorphisms in amplicons, if more than one signal sequences were used.

Those skilled in the art will appreciate that the described signal primer and opposite primer and methods can be used to amplify two or more different target nucleic acid molecules (such as at least 2, at least 3, at least 4, or even at least 5 different nucleic acid sequences) in the same amplification reaction. In particular examples, two or more different primers containing same signal sequence, and labeled detection probes contain the same corresponding signal sequence, are used. In other examples, the different signal sequence are contained in at least two different sequence-specific primers, wherein the resulting amplicons are differentiated, for example by labeled detection probes containing their corresponding signal sequences.

Since the signal sequence doesn't need to be exist in, or related to the target, any sequence fragments that works well in one detection system can be used in any other detection system. This provides significant variety and flexibility in designing signal sequences.

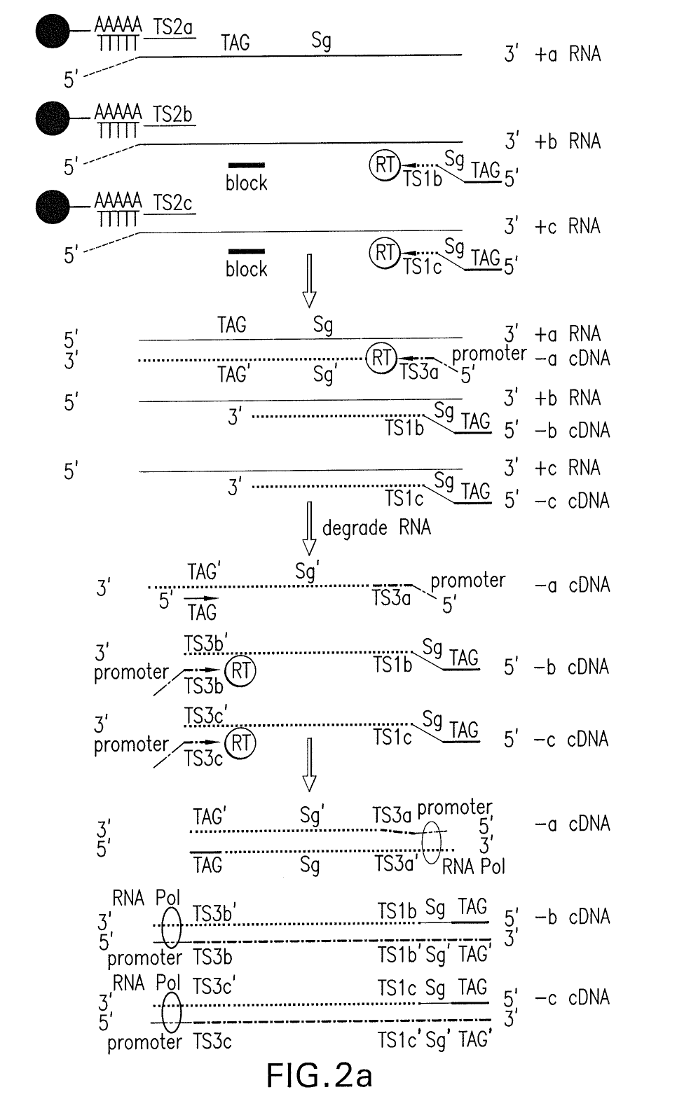

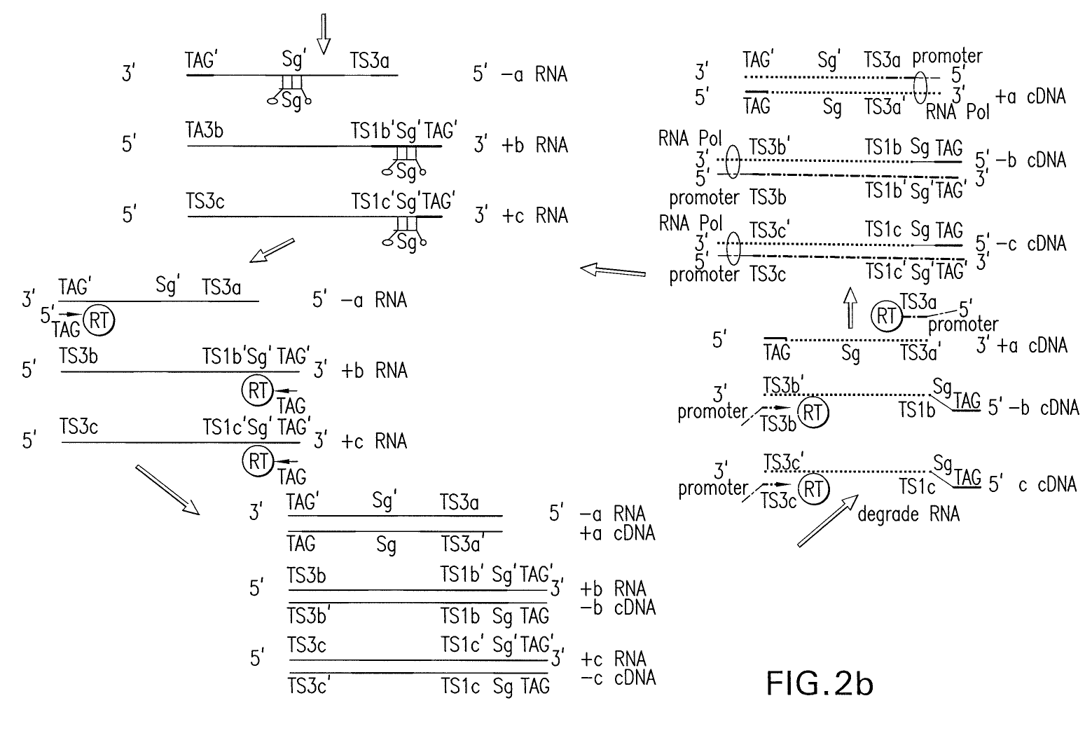

There are several methods which can be used to obtain one or more signal sequences. One method for designing a signal sequences is to select from systems that have been previously optimized. This can be done from published literatures or their own experiences. For example, from an optimized real-time HIV detection system using molecular beacon technology, the HIV detection probe sequences can be used as “signal sequences” to design new systems for HBV or HCV detection (see, e.g., FIG. 2). In this case, the design and optimization of HBV or HCV specific detection probes are no longer needed.

A similar method for obtaining a signal sequences is using a well characterized sequence as the signal sequence. For example, one can use an optimized detection probe sequences used in real-time HIV detection to detect the presence or the nucleic acid level of a PCA3 (a cancer marker) in a sample.

Another method for obtaining a signal sequence is to select from computer generated random sequences. For example to obtain a TaqMan or molecular beacon sequences by this method. Once such a sequence is identified, it can be used in many different detection systems using the methods described in this invention.

Another method is to design such a signal sequence from scratch. Again, once one or more of the detection probe sequences are obtained, it can then be to detect many different targets, using the method described in this invention.

The simplicity and variability methods of obtain such a signal sequence significantly facilitate the primer and detection probe design process in various nucleic acid amplification and detection technologies. For example, certain target nucleic acid molecules, such as small RNA or iRNA targets, where the available sequences for primer and detection probe design are limited; another example would be to detect certain group of bacterial or virus where highly conserved region for traditional primer-detection probe-primer design scheme is not feasible. With the method described in the present invention, which uses signal sequences from other systems, such a design and detection is now simple and feasible.

### 5.2 METHODS OF THE INVENTION

The described signal sequences can be used in any nucleic acid amplification reaction to determine whether a particular target nucleic acid sequence is present, such as a DNA or RNA molecule For example, methods are described for detecting a target nucleic acid molecule. In particular examples, the method includes incubating a sample with a signal sequence linked to a target-specific sequence to form a “signal primer”, or linked to a target-specific sequence and a promoter sequence to form a “opposite primer”. In some examples, one or both the signal primer and the opposite primer contain one or more TAG sequences.

Any primer extension amplification method can be used. Particular examples include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. (Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996). For example, TMA can be performed using the signal sequence-containing detection probes and signal sequence-containing primers (either T7 or non T7, see picture 2, 3, &8) described herein.

A change in detectable signal from the label on the signal sequence containing detection probe is monitored, wherein a change in signal indicates the presence of the target nucleic acid sequence, and wherein no significant change in signal indicates that the target nucleic acid molecule is not present in the sample. The change in signal can be compared to a signal present earlier, such as prior to nucleic acid amplification. The detectable signal changes in a predictable manner that permits determination of whether or not a target nucleic acid sequence is present in a sample, and in some examples, quantization of an amount of target nucleic acid sequence is present in a sample.

In some examples, the change in signal is monitored during the amplification reaction, for example in real-time as the amplicons are formed. For example, the label present on the signal sequence containing detection probe will generate a significant signal or not, when the detection probe are freely floating in the nucleic acid amplification reaction mixture. During nucleic acid amplification, when polymerase creates nucleic acid amplicons, the detection probe, hybridize with the amplicon. The signal from the label will increase or decrease due to its hybridization with the amplicon molecule. As more amplicons are produced during nucleic acid amplification, the overall signal of the reaction mixture will increase or decrease. The change in signal can be monitored using any commercially available system. This change in signal permits detection of a target nucleic acid sequence in the reaction.

In other or additional examples, the change in signal is monitored after the amplification, for example by exposing the resulting amplicons to a melting procedure to denature the double-stranded amplicons. During the denaturation, a change in signal is detected. The resulting signal peaks, such as fluorescence peaks, can differentiate polymorphisms in the nucleic acid amplicons. Therefore, melting curve analysis can be used to confirm the presence of a target nucleic acid sequence, and can also be used to distinguish polymorphisms in amplicons.

In other examples, the change in signal that is monitored during the amplification reaction is an increase in fluorescence. In this example, the detection probe includes a stem and loop structure, wherein the stem represents the non-target nucleic acid sequence (which contains a single internal fluorescent label that remains quenched with its complementary stem part). However, during target-dependent synthesis, a complementary strand is synthesized and the detection probe hybridizes to the amplicon.

In other examples, the signal sequence containing detection probe are structured such that the fluorophore is quenched by another quenching fluorophore and emits fluorescence upon hybridization to the complementary sequence of the signal sequence attached to the signal primer. Examples of these types of detection probe structures include Molecular Beacons (Tyagi et al., Nature Biotech. 14.303-8, 1996; U.S. Pat. No. 5,989,823, the disclosure of which is incorporated herein by reference) and molecular torches, cleavable beacons.

Methods of detecting a target nucleic acid molecule following nucleic acid amplification are provided. The methods include incubating a sample containing or thought to contain the target nucleic acid molecule with a forward primer and a reverse primer that are specific for the target nucleic acid molecule. Either the forward primer or the reverse primer is linked at its 5′-end to the 3′-end of a optima tag, under conditions sufficient to allow amplification of the target nucleic acid molecule (such as real-time PCR conditions). However, in some examples, both the forward and the reverse primer are linked at their 5′-end to the 3′-end of a universal tag. In some reactions, such as reverse TMA, one of the primer can be 3′ blocked (i.e., only one extendable primer is needed to start the primer extension reaction). The amplification results in the generation of a complementary sequence to the labeled signal sequence. The amplicons is exposed to conditions that permit hybridization of the amplicons with the labeled signal sequence. This results in a change in detectable signal, for example relative to the detectable signal from the label upon the formation of double stranded DNA. A change in signal indicates that the target nucleic acid molecule is present in the sample, and no significant change in signal indicates that the target nucleic acid molecule is not present in the sample.

In addition to determining whether a particular target nucleic acid molecule is present, the method can further include quantifying the target nucleic acid molecule. In one example quantitation includes comparing a signal to an amount of signal from a known amount of nucleic acid.

Samples containing nucleic acid molecules can be obtained from any appropriate specimen, for instance, urine, blood or blood-fractions (such as serum). Techniques for acquisition of such samples are well known in the art (for example see Schluger et al. J. Exp. Med. 176:1327-33, 1992, for the collection of serum samples). Serum or other blood fractions can be prepared in the conventional manner. For example, about 200 μL of serum can be used for the extraction of DNA or RNA for use in amplification reactions.

Once a sample has been obtained, the sample can be used directly, concentrated (for example by centrifugation or filtration), purified, or combinations thereof. In one example, DNA is prepared from the sample, yielding a nucleotide preparation that is accessible to, and amenable to, nucleic acid amplification. Similarly, RNA can be prepared using a commercially available kit (such as the RNeasy Mini Kit, Qiagen, Valencia, Calif.).

The signal primer and detection means described above are useful for the selective amplification and detection of one or more DNA or RNA nucleic acid targets, and can be used in the many conventional amplification systems or variations thereof (e.g., TMA, PCR, RT-PCR, etc.). The combination offers many advantages including, but not limited to, increased flexibility in primer sequence and probe sequence design for single nucleic acid target amplification and detection, as well as reduction in number of primers and detection probes required for multiple nucleic acid targets amplification and detection.

The following sections describe and illustrate non-limiting examples of possible use of the tagged primer and tagged probe of the invention in amplification reactions.

The presence of a target nucleic acid in a sample can be determined. In addition, changes in expression of one or more target nucleic acids can also be determined. The present disclosure is not limited to particular methods of detection. Any method of detecting a nucleic acid molecule can be used, such as physical or functional assays.

In one example, the described method includes amplifying a target nucleic acid molecule using primers containing signal sequence. For example, a target nucleic acid molecule can be amplified using a signal primer containing a signal sequence, an opposite primer, and the amplified products (“amplicons”) can be detected using a labeled detection probe also containing the signal sequence, such as a molecular beacon. If the target nucleic acid molecule is present, an amplicon containing the complementary sequence to the signal sequence will be generated. In a particular example, the methods are useful for amplification and detection of a target nucleic acid sequence obtained from a subject, such as a subject having or suspected of having an infection or a disease.

Detecting a hybridized complex has been previously described. In one example, detection includes detecting one or more labels hybridized with the amplicons. In particular examples, the method further includes quantification, for instance by determining the amount of hybridization, for example relative to a control (such as a known amount of the target nucleic acid molecule).

5.2.1 TMA

Typically, a transcription-mediated amplification (TMA) system useful for the selective amplification and detection of a single nucleic acid target includes the following steps:
Step 1: binding to a target RNA a first primer (Primer 1) which comprises a promoter sequence and a sequence (TS1) that binds to a region (TS1′) of the target RNA, and synthesizing a first DNA copy of the target RNA using reverse transcriptase (RT);
Step 2: forming a target RNA:DNA duplex;
Step 3: degrading the target RNA using RNAse H activities of RT;
Step 4: binding to the first DNA copy a second primer (Primer 2) which comprises a sequence (TS2) that binds to a region (TS2′) of the first DNA copy, and synthesizing a second DNA copy using RT;
Step 5: forming a double-stranded DNA with a promoter sequence;
Step 6: transcribing RNA from the double-stranded DNA using RNA polymerase (RNA-Pol);
Step 7: producing 100-1000 copies of RNA amplicons;
Step 8: binding Primer 2 to each RNA amplicon and synthesizing a third DNA copy;
Step 9 forming a RNA amplicon:DNA duplex;
Step 10: degrading the RNA amplicon using RNAse H activities of RT; and
Step 11: binding Primer 1 to the third DNA copy and synthesizing a fourth DNA copy.

The autocatalytic cycle (e.g., Steps 5-11) will repeat, resulting in multiple RNA amplicons, and the RNA amplicons can be detected using a detection probe that comprises a sequence (TS3) which binds specifically to a region (TS3′) of the RNA amplicon. In such a system, two target-specific primers and a target-specific probe are required for each nucleic acid target.

Primer 2 can be used in Step 1 and Primer 1 can be used in Step 4 to produce RNA amplicons having the same polarity (sense) as the target RNA.

A TMA system useful for the selective amplification and detection of multiple nucleic acid targets typically include the same steps as those involved in the selective amplification and detection of a single nucleic acid target, except requiring an increased number of primers and probes. In conventional TMA, two target-specific primers and one target-specific probe are required for the selective amplification and detection of each nucleic acid target. Thus, a TMA system for the selective amplification and detection of two nucleic acid targets will require a total of four primers and two probes, a TMA system for the selective amplification and detection of three nucleic acid targets would require a total of six primers and three probes, a TMA system for the selective amplification and detection of four nucleic acid targets would require a total of eight primers and four probes, and so forth.

The number of primers can be reduced by introducing into Primer 1a first universal (U1) sequence (e.g., a TAG sequence) between the promoter sequence and the sequence that binds to the target RNA, and introducing into Primer 2 a second universal (U2) sequence upstream of the sequence that binds to the first DNA copy. As a result, the RNA amplicons generated in Step 7 would comprise a U1 sequence, the T×S1 sequence downstream of the U1 sequence, a TxS2′ sequence complementary to the TxS2 sequence and downstream of the T×S1 sequence, and a U2′ sequence complementary to the U2 sequence and downstream of the TxS2′ sequence. The term “Tx” is used herein to denote different nucleic acid targets (e.g., Ta, Tb, Tc, etc.). In subsequent Steps 8 and 11, the same set of primers (i.e., a universal non-promoter primer consisting of the U2 sequence, and a universal promoter primer consisting of (i) a promoter sequence and (2) the U1 sequence downstream of the promoter sequence) can then be used for different nucleic acid targets, instead of one unique set of primers per nucleic acid target. While the use of such universal sequences reduces the number of primers, the number of probes required in the multiplex detection assays remain the same, i.e., one probe per nucleic acid target. Plus, the fact that the probe sequence will need to hybridize to a unique region on each nucleic acid target could be a burdensome design requirement, as well as an additional source of false positives, since it might share sequence homology with some of the bacterial or viral enzymes used during the amplification process.

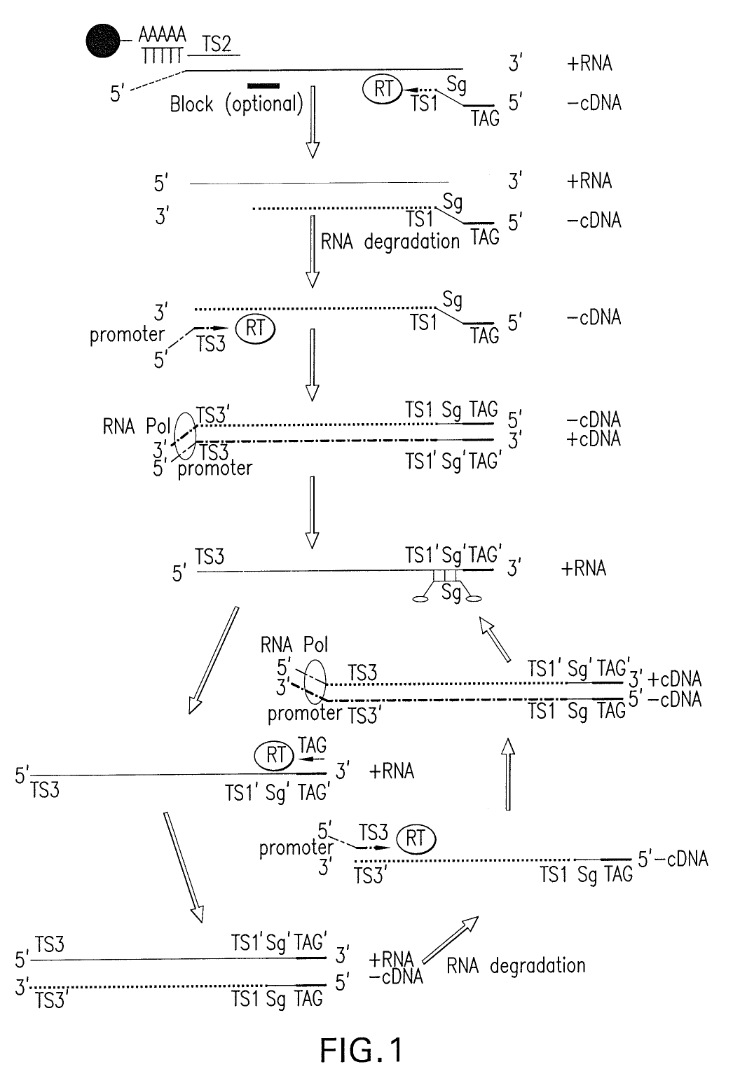

As shown in FIG. 1, the signal primer and detection means of the invention offers alternative methods for detecting a nucleic acid target in a TMA reaction. First, a sample comprising or is suspected of comprising a nucleic acid target is mixed with a signal primer and a capture moiety of the invention, wherein the signal primer comprises (i) a first target-specific (TS1) sequence which hybridizes specifically to the nucleic acid target, and (ii) a signal (Sg) sequence upstream of the TS1 sequence, wherein the signal sequence is not found in the nucleic acid target or its complementary sequence, and (iii) a TAG sequence upstream of the signal sequence, wherein the TAG sequence is not found in the signal sequence, the nucleic acid target, or their respective complementary sequences; and the capture moiety comprises a solid support linked with a second target-specific (TS2) sequence which hybridizes specifically to the nucleic acid target, wherein the TS1 and TS2 sequences hybridize to non-overlapping regions of the nucleic acid target; wherein said mixing is conducted under a condition for the TS1 and TS2 sequences to hybridize to the nucleic acid target and form a complex comprising the nucleic acid target hybridized with the signal primer and the capture moiety. Second, the complex is isolated or purified by separating it from excess signal primer not hybridized to the nucleic acid target. Third, the isolated or purified complex is mixed with an opposite primer and a TAG primer in a mixture, wherein the TAG primer comprises the TAG sequence, and the opposite primer comprises (i) a third target-specific (TS3) sequence selected from a region of the nucleic acid target that is upstream of the region to which the TS1 sequence hybridizes, and (ii) a promoter (Pm) sequence upstream of the TS3 sequence. Fourth, the nucleic acid target present in Step (c) is amplified to generate a plurality of an amplified RNA product that comprises, in the direction from the 5′-end to the 3′-end, the TS3 sequence, the complementary sequence to the TS1 sequence, the complementary sequence to the signal sequence, and the complementary sequence to the TAG sequence. Finally, the presence of the amplified RNA product is detected with a detection means which is capable of detecting the presence of the complementary sequence to the signal sequence, wherein the detection means is introduced into the mixture during or after Step (c).

As shown in FIG. 2, the signal primer and detection means of the invention offers alternative methods for detecting two or more different nucleic acid targets in a TMA reaction. First, a sample comprising or is suspected of comprising at least a first nucleic acid target (Ta) and a second nucleic acid target (Tb) is mixed with a Ta-specific signal primer, a Ta-specific capture moiety, a Tb-specific signal primer and a Tb-specific capture moiety, wherein the Ta-specific signal primer comprises (i) a first Ta-specific (TS1a) sequence which hybridizes specifically to the Ta nucleic acid target, and (ii) a signal (Sg) sequence upstream of the TS1a sequence, wherein the signal sequence is preferably not found in the Ta nucleic acid target, the Tb nucleic acid target, or their respective complementary sequences, and (iii) a TAG sequence upstream of the signal sequence, wherein the TAG sequence is preferably not found in the signal sequence, the Ta nucleic acid target, the Tb nucleic acid target, or their respective complementary sequences, and the Ta-specific capture moiety comprises a solid support linked with a second Ta-specific (TS2a) sequence which hybridizes specifically to the Ta nucleic acid target, wherein the TS1a and TS2a sequences hybridize to non-overlapping regions of the Ta nucleic acid target, and the Tb-specific signal primer comprises (i) a first Tb-specific (TS1b) sequence which hybridizes specifically to the Tb nucleic acid target, and (ii) the signal sequence upstream of the TS1b sequence, and (iii) the TAG sequence upstream of the signal sequence, and the Tb-specific capture moiety comprises a solid support linked with a second Tb-specific (TS2b) sequence which hybridizes specifically to the Tb nucleic acid target, wherein the TS and TS2ba sequences hybridize to non-overlapping regions of the Tb nucleic acid target; wherein said mixing is conducted under a condition for the TS1a and TS2a sequences to hybridize to the Ta nucleic acid target and form a Ta complex comprising the Ta nucleic acid target hybridized with the Ta-specific signal primer and the Ta-specific capture moiety and for the TS and TS2b sequences to hybridize to the Tb nucleic acid target and form a Tb complex comprising the Tb nucleic acid target hybridized with the Tb-specific signal primer and the Tb-specific capture moiety. Second, the Ta and Tb complexes is isolated or purified by separating them from excess Ta specific signal primer not hybridized to the Ta nucleic acid target and from excess Tb-specific signal primer not hybridized to the Tb nucleic acid target. Third, the isolated or purified Ta and Tb complexes are mixed with a Ta-specific opposite primer, a Tb-specific opposite primer and a TAG primer in a mixture, wherein the TAG primer comprises the TAG sequence, the Ta-specific opposite primer comprises (i) a third Ta-specific (TS3a) sequence selected from a region of the Ta nucleic acid target that is upstream of the region to which the TS1a sequence hybridizes, and (ii) a first promoter (Pma) sequence upstream of the TS3a sequence, and the Tb-specific opposite primer comprises (i) a third Tb-specific (TS3b) sequence selected from a region of the Tb nucleic acid target that is upstream of the region to which the TS1b sequence hybridizes, and (ii) a second promoter (Pmb) sequence upstream of the TS3b sequence. Fourth, the Ta and Tb nucleic acid targets present in Step (c) are amplified to generate a plurality of an amplified Ta RNA product and an amplified Tb RNA product, wherein the amplified Ta RNA product comprises, in the direction from the 51-end to the 3′-end, the TS3a sequence, the complementary sequence to the TS1a sequence, the complementary sequence to the signal sequence, and the complementary sequence to the TAG sequence, and the amplified Tb RNA product comprises, in the direction from the 5′-end to the 3′-end, the TS3b sequence, the complementary sequence to the TS1b sequence, the complementary sequence to the signal sequence, and the complementary sequence to the TAG sequence. Finally, the presence of the amplified Ta and Tb RNA products are detected with a detection means which is capable of detecting the presence of the complementary sequence to the signal sequence, wherein the detection means is introduced into the mixture during or after Step (c).

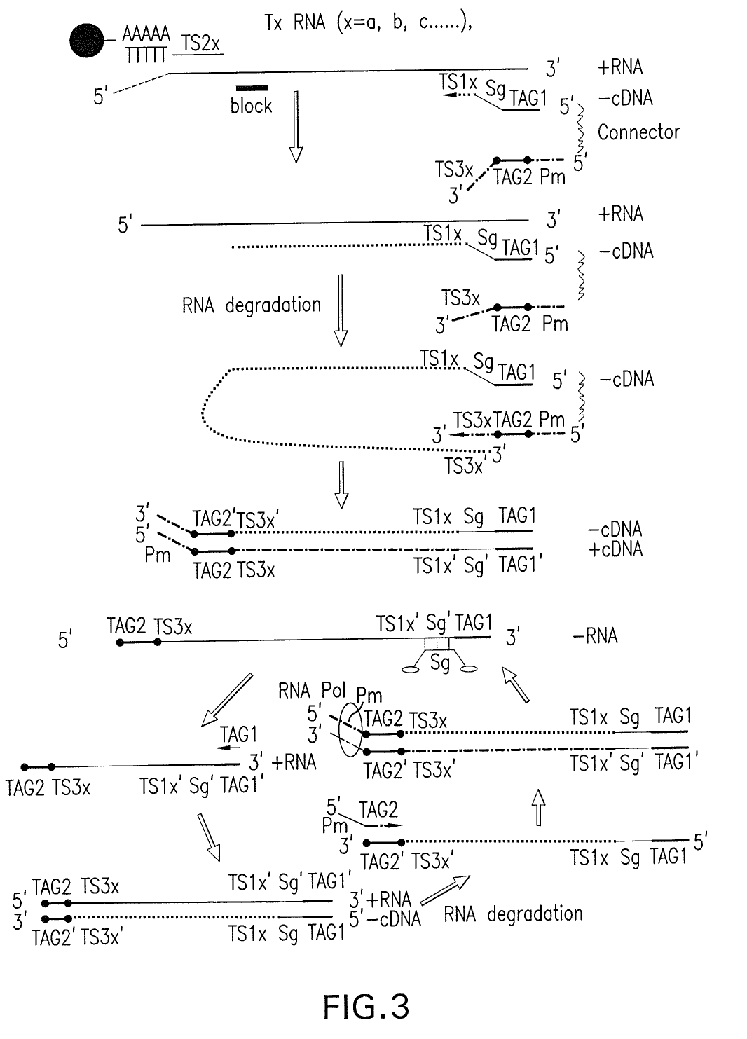

As illustrated in FIG. 2, in the above described multiplex amplification, a TAG primer and a plurality of target-specific opposite primers are used to generate amplicons containing the complementary sequence of the signal sequence for detection. In a further preferred embodiment, as illustrated in FIG. 3, a second TAG primer is used in place of the plurality of opposite primers for generating the amplicons. This protocol involves the following oligonucleotides: (1) a target-specific capture moiety for each target (+) comprising a target-specific sequence (TS2x, x=a, b, . . . ) (−); (2) a target-specific dual TAG primer comprising two primers (i.e., a target-specific signal primer and a target-specific opposite primer) connected together at their respective 5′-ends by a connector; (3) a non-promoter TAG primer comprising the first TAG sequence; and (4) a promoter TAG primer comprising the second TAG sequence and a promoter sequence upstream of the second TAG sequence. The target-specific signal primer for each target comprises, from the 5′-end to the 3′-end, a first TAG (TAG1) sequence, the signal sequence, and a target-specific sequence (TS1x, x=a, b, . . . ) (−). The target-specific opposite primer for each target comprises, from the 5′-end to the 3′-end, the promoter sequence, a second TAG (TAG2) sequence, and a target-specific sequence (TS3x, x=a, b, . . . ) (+). Optionally, a block oligonucleotide is used in the method.

In certain embodiments, the connector could be an oligoethylene glycol bridge or other linkers known to those skilled in the art.

In certain embodiments, the TAG1 primer comprises the TAG1 sequence (−).

In certain embodiments, the TAG2 primer comprises the TAG2 sequence (+) and a promoter sequence upstream of the TAG2 sequence.

In this protocol, both the dual TAG primer and the capture moiety are incorporated into the complex with the target nucleic acid by hybridization. Following extension from the signal primer, the opposite primer hybridizes to the newly synthesized complementary strand of the target nucleic acid (“-cDNA”) and allow the synthesis of +cDNA. The promoter in the opposite primer then drives transcription to make a RNA amplicon comprising, from the 5′-end to the 3′-end, the TAG2 sequence, the TS3× sequence, the complementary sequences to the TS1x sequence, the signal sequence, and the TAG1 sequence. The TMA reaction thereafter is sustained by the pair of TAG1 primer and TAG2 primer to make additional RNA amplicon for detection by a detection probe, preferably by a detection probe containing the signal sequence.

Regardless the number of different target nucleic acids (e.g., 77 subtypes of HPV) for detection), this protocol can reduce the number of primers in the amplification reagents to two, namely, the TAG1 primer and TAG2 primer.

### 6. EXAMPLES

Examples are provided below illustrating certain aspects and embodiments of the invention. The examples below are believed to accurately reflect the details of experiments actually performed, however, it is possible that some minor discrepancies may exist between the work actually performed and the experimental details set forth below which do not affect the conclusions of these experiments or the ability of skilled artisans to practice them. Skilled artisans will appreciate that these examples are not intended to limit the invention to the specific embodiments described therein. Additionally, those skilled in the art, using the techniques, materials and methods described herein, could easily devise and optimize alternative amplification systems for carrying out these and related methods while still being within the spirit and scope of the present invention.

The following experiments were conducted to evaluate whether the use of a pair of signal primer and detection probe which each comprises a signal sequence would permit the selective amplification and detection of target nucleic acid sequence contributed by the nucleic acid sample of interest, while not amplifying and detecting target nucleic acid sequence contributed by sources other than the nucleic acid sample of interest.

### 6.1 Example 1

### Selective Amplification of U. urealyticum rRNA with a Pair of Signal Primer and Molecular Beacon Detection Probe, Each Comprising a Signal Sequence

6.1.1 Materials and Methods

In this example, the target nucleic acid is U. urealyticum rRNA (UU rRNA).

Reagents and protocol conditions used in the performed experiments, as well as a discussion of the results and conclusions of the experiments, are set forth below.

Oligonucleotides Used:

Signal Primer (including TAG sequence, signal sequence, and TS1 sequence): TCACAATTTTAAAAGAAAAGGG- [symbol] [symbol] -TACACTCTA GGTTTACAGTT (SEQ ID NO:1); 10 pmol/rxn, the underlined is the signal sequence.

Tag Primer:

(SEQ ID NO: 2)

TCACAATTTTAAAAGAAAAGGG; 7.5 pmol/rxn.

Opposite Primer (including promoter sequence+TS3 sequence): [symbol] [symbol] [symbol] -GAGAGAGCGCAGGCGGGTTTGTAAGTT TGGTA (SEQ ID NO:3); 7.5 pmol/rxn, the underlined is the promoter sequence.

Block Oligonucleotide:

cgcucguuuuacgcccagua (SEQ ID NO:4); 0.8 pmol/rxn. Nucleosides in lower case mean 2′-OME bases.

Capture Oligonucleotide (including TS2 sequence):

(SEQ ID NO: 5)

guauuaccgcggcugcuggc-
TTTAAAAAAAAAAAAAAAAAAAAAAAAAAAAAA; 15 pmol/rxn.

Detection Probe (molecular beacon, including the signal sequence):

ccgag- [symbol] [symbol] -ucgg (SEQ ID NO:6); 5 pmol/rxn, the underlined is the signal sequence.

Reagents Used:

U.urealyticum overnight culture in urea broth medium.

The “Amplification Reagent” or “AMP Reagent” comprised 26 mM Trizma base buffer, 25 mM MgCl₂, 213 mM KCl₂, 3.33% (v/v) glycerol, 0.05 mM zinc acetate, 0.76 mM DATP, 0.76 mM dCTP, 0.76 mM dGTP, 0.76 mM dTTP, 4.0 mM ATP, 4.0 mM CTP, 4.0 mM GTP, and 4.0 mM UTP, pH 8.0 at 22° C.

Enzyme Reagent. The “Enzyme Reagent” comprised 70 mM N-acetyl-L-cysteine, 10% (v/v) TRITON X-102 detergent, 16 mM HEPES, 3 mM EDTA, 20 mM Trizma base buffer, 50 mM KCl₂, 20% (v/v) glycerol, 165.6 mM trehalose, pH 7, and containing 250 RTU/μl Moloney murine leukemia virus (“MMLV”) reverse transcriptase and 140 U/μL T7 RNA polymerase, where one unit (i.e., RTU or U) of activity is defined as the synthesis and release of 5.75 fmol cDNA in 15 minutes at 37° C. for MMLV reverse transcriptase, and the production of 5.0 fmol RNA transcript in 20 minutes at 37° C. for T7 RNA polymerase.

Wash Buffer. The “Wash Buffer” comprised 10 mM HEPES, 1 mM EDTA, 0.3% (v/v) ethyl alcohol, 150 mM NaCl, and 0.1% (w/v) sodium dodecyl sulfate, pH 7.5.

Target Capture Reagent. The “Target Capture Reagent” comprised capture oligonucleotide: 15 pmol/rxn; signal primer including TAG sequence, signal sequence and TS1 sequence: 10 pmol/rxn; block oligonucleotide: 0.8 pmol/rxn; HEPES, Free Acid, 250 mM; Lithium Hydroxide, Monohydrate: 310 mM; Lithium Chloride: 1880 mM; EDTA: 100 mM; Lithium Lauryl Sulfate: 110.2 mM; Ammonium Sulfate: 37.5 mM; Magnetight dT25 Magnetic Particles: 0.075 ug/uL.

Detection Machine Used:

ABI 7300 was used as the detection machine.

Detailed Protocol:
Place the desired number of 1.5 ml eppendorf tubes in the designated rack.
Add 100 μl Target Capture Reagent (TCR) to each tube.
Add 400 μl of sample to each tube.
Vortex on high speed for 15 seconds.
Place the tubes in a 60° C. water bath and incubate for 1 to 10 minutes.
Incubate at room temperature for 1 to 10 minutes.
Place tubes on magnetic base for 3 minutes to separate the beads from the solution.
Aspirate the supernatant.
Add 1 ml of Wash Buffer (WB).
Vortex on high speed for 15 seconds.
Place the tubes on magnetic base for 3 minutes to separate the beads from the solution.
Aspirate the supernatant.
Repeat steps 9-12 (some assays do not require this additional wash).
Add 30 μl of Amp Reagent to each tube
Vortex on high speed for 15 seconds.
Transfer the contents of each tube to individual wells of a 96 well microtiter plate.
Cover the plate with a clear sealing card.
Incubate the 96 well microtiter plate at 60° C. for 0 to 5 minutes.
Incubate the 96 well microtiter plate at 42° C. for 2 to 5 minutes.
Add 10 μl of Enzyme Reagent to each of the wells
Immediately place the plate into ABI7300 (isothermal incubation at 42° C.) and measure fluorescence of each well every 1 minute.

6.1.2 Results

A target capture step was performed for binding U. urealyticum rRNA and removing unhybridized tagged priming oligonucleotide and block oligonucleotide. After the target capture step, an AMP Reagent was added, with the AMP Reagent containing a tag priming oligonucleotide and the detection probe with the same signal sequence as included in the signal primer. No signal primer was included in this step.

The UU AMP Reagent contained 7.5 pmol opposite primer, 7.5 pmol TAG primer and 5 pmol molecular beacon per reaction.

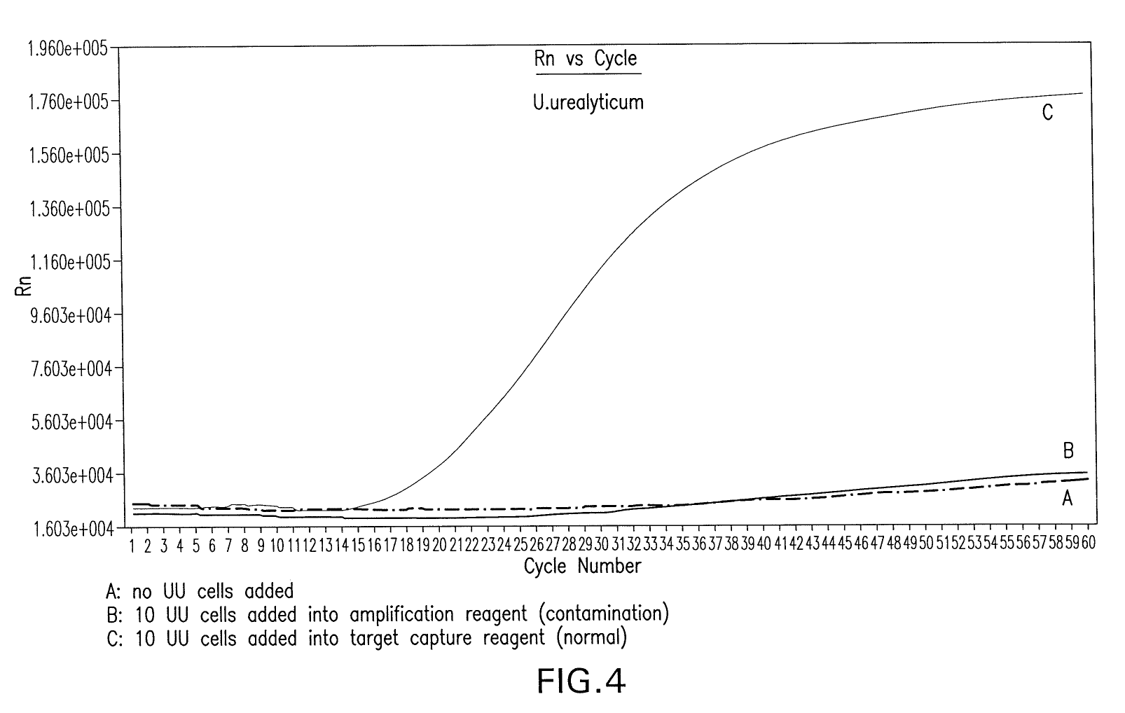

The first set of experiments compared the results of reactions in which no UU cells were spiked into the Target Capture Reagent or AMP Reagent with the results of reactions in which 1×10³ cells of UU were spiked into the Target Capture Reagent. FIG. 4 shows the raw curves of amplifications in which no target or 10 UU cells was spiked into the AMP Reagent. Line A shows that there was no detectable amplification when no UU cells were spiked into the Target Capture Reagent. Line B shows that there was no detectable amplification when 10 UU cells were spiked into the AMP Reagent. Line C shows detectable amplification when 10 UU cells were spiked into the Target Capture Reagent. This indicates that the detection method will not pick up target introduced in the amplification phase, i.e., potential contamination, but is effective in detecting target in the capturing phase, i.e., target from test samples.

### 6.2 Example 2

### Use of Signal Primer Allows Discrimination Between Sample-Derived Templates and Exogenous Templates

6.2.1 Materials and Methods

In this example, the target nucleic acid is E. coli 16s rRNA. This example describes a procedures for amplifying E. coli 16s rRNA nucleic acid. The procedure used a signal primer, a block oligonucleotide, an opposite primer and a detection probe, which also contains a signal sequence. Neither of the signal sequence and the TAG primer hybridizes to the E. coli rRNA nucleic acid target or the complement thereof.

In this procedure, a signal primer and target capture step were employed for performing amplification reactions containing either 0, 10³ or 10⁵ E. coli cells.

Oligonucleotides used in the procedure are indicated below. The molecular beacon detection probe was added in the AMP reagent. Following target capture, signal primer that was not hybridized to template nucleic acid was removed from the system by wash steps. The complex that included the rRNA template and the signal primer remained were captured on super-paramagnetic particles.

Oligonucleotides Used:

Signal Primer (including TAG sequence, signal sequence, and TS1 sequence): TCACAATTTTAAAAGAAAAGGG- [symbol] [symbol] -ATGTCAAGAGT AGGTAAGGTTCTTCGCG (SEQ ID NO:7); 10 pmol/rxn, the underlined is the signal sequence.

TAG Primer:

(SEQ ID NO: 2)

TCACAATTTTAAAAGAAAAGGG; 7.5 pmol/rxn.

Opposite Primer (including promoter oligonucleotide+TS3 sequence):

[symbol]

[symbol]

[symbol]

[symbol]

AAIGAATTGACGIGG (SEQ ID NO:8); 7.5 pmol/rxn, the underlined is the promoter sequence. “I” denotes Inosine. AAAIGAATTGACGIGG (SEQ ID NO. 9) was found to hybridize to the rRNA of a group of bacteria, including Escherichia coli, Citrobacter freundii, Klebsiella pneumoniae, Enterobacter cancerogenus, Hafnia alvei, Enterobacter cloacae, Pseudomonas aeruginosa, Pseudomonas fluorescens, Pseudomonas fuscovaginae, Pseudomonas grimontii, Staphylococcus aureus, Staphylococcus auricularis, Staphylococcus carnosus, Staphylococcus epidermidis, and Staphylococcus condimenti. Therefore, an oligonucleotide comprising SEQ ID NO. 9 can be used to detect the presence of nucleic acid from bacteria selected from the group consisting of, but not limited to the following bacteria: Escherichia coli, Citrobacter freundii, Klebsiella pneumoniae, Enterobacter cancerogenus, Hafnia alvei, Enterobacter cloacae, Pseudomonas aeruginosa, Pseudomonas fluorescens, Pseudomonas fuscovaginae, Pseudomonas grimontii, Staphylococcus aureus, Staphylococcus auricularis, Staphylococcus carnosus, Staphylococcus epidermidis, and Staphylococcus condimenti.

Block Oligonucleotide:

(SEQ ID NO: 10)

cauuugaguuuuaaccuugc; 0.4 pmol/rxn.
Capture Oligonucleotide (including TS2 Sequence):

(SEQ ID NO: 11)

ccaggcggucgacuuaacgc-
TTTAAAAAAAAAAAAAAAAAAAAAAAAAAAAAA; 15 pmol/rxn.

Detection Probe (molecular beacon, including the signal sequence):

ccgag- [symbol] [symbol] -ucgg (SEQ ID NO:6); 5 pmol/rxn, the underlined is the signal sequence.

6.2.2 Results

The target capture reagent, AMP reagent, enzyme reagent and the experimental protocol are the same as described in example 1. In target capture step, the specific capture oligonucleotide was added a final concentration of 15 pmol/rxn. Amplification solution also contained opposite primer, molecular beacon detection probe and TAG primer. Dilutions of the E. coli culture were prepared in water. 0, 10³ or 10⁵ copies of the E. coli cells were used for each reaction.

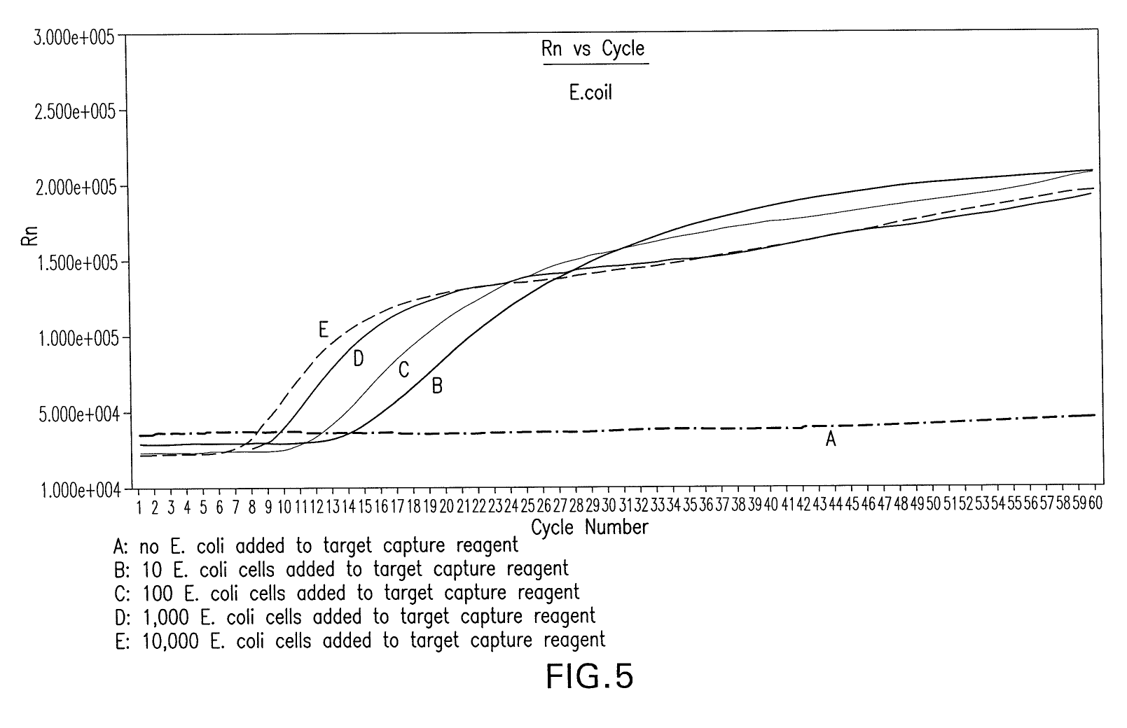

FIG. 5 shows the quantitative raw curve of amplification when different number of E. coli cells (from 0 to 10,000 cells per reaction, as indicated) were added into the target capture reagent. The negative control (neg) (Line A) where no E. coli cells were added into the target capture reagent indicate that no amplification were detected.

This experiment shows that the method of the invention is capable of measuring the emergence of the signal which is inversely proportional to the level of target introduced at the target capturing step, i.e., target from test samples.

### 6.3 Example 3

### Background Comparison: Signal Primer & Detection Probe Pair in comparison with Target-Specific Detection Probe

6.3.1 Materials and Methods

In this example, the target nucleic acid is E. coli 16s rRNA. The experiment was conducted to compare the level of background signal between a design of the current invention and that of an existing technology (using a detection probe against a region of E. coli rRNA).

Materials, and results using current invention are listed below. The experimental protocol and other reagents are the same as described in example 2.

Oligonucleotides Used:

Signal Primer (including TAG sequence, signal sequence, and TS1 sequence): TCACAATTTTAAAAGAAAAGGG- [symbol] [symbol] -ATGTCAAGAGT AGGTAAGGTTCTTCGCG (SEQ ID NO:7); 10 pmol/rxn, the underlined is the signal sequence.

TAG Primer:

(SEQ ID NO: 2)

TCACAATTTTAAAAGAAAAGGG; 7.5 pmol/rxn.

Opposite Primer (including promoter sequence and TS3 sequence):

[symbol]

[symbol]

[symbol]

[symbol]

CGCAAGGTTAAAACTCAAA (SEQ ID NO:12); 7.5 pmol/rxn.

Block Oligonucleotide:

(SEQ ID NO: 13)

uugcggccguacuccccaagg; 0.4 pmol/rxn.
Capture Oligonucleotide (including TS2 sequence):

(SEQ ID NO: 14)

uagcuccggaagccacgccu-
TTTAAAAAAAAAAAAAAAAAAAAAAAAAAAAAA; 15 pmol/rxn.

Detection Probe (molecular beacon, including the signal sequence):

ccgag- [symbol] [symbol] -ucgg (SEQ ID NO:6); 5 pmol/rxn, the underlined is the signal sequence.

Alternate Detection Probe:

ccgag- [symbol] -cucgg (SEQ ID NO:15); the underlined is a target-specific sequence.

Alternate Primer: TCACAATTTTAAAAGAAAAGGG-ATGTCAAGAGT AGGTAAGGTTCTTCGCG (SEQ ID NO:16). SEQ ID NO:16 is SEQ ID NO:7 minus the signal sequence in the middle.

6.3.2 Results

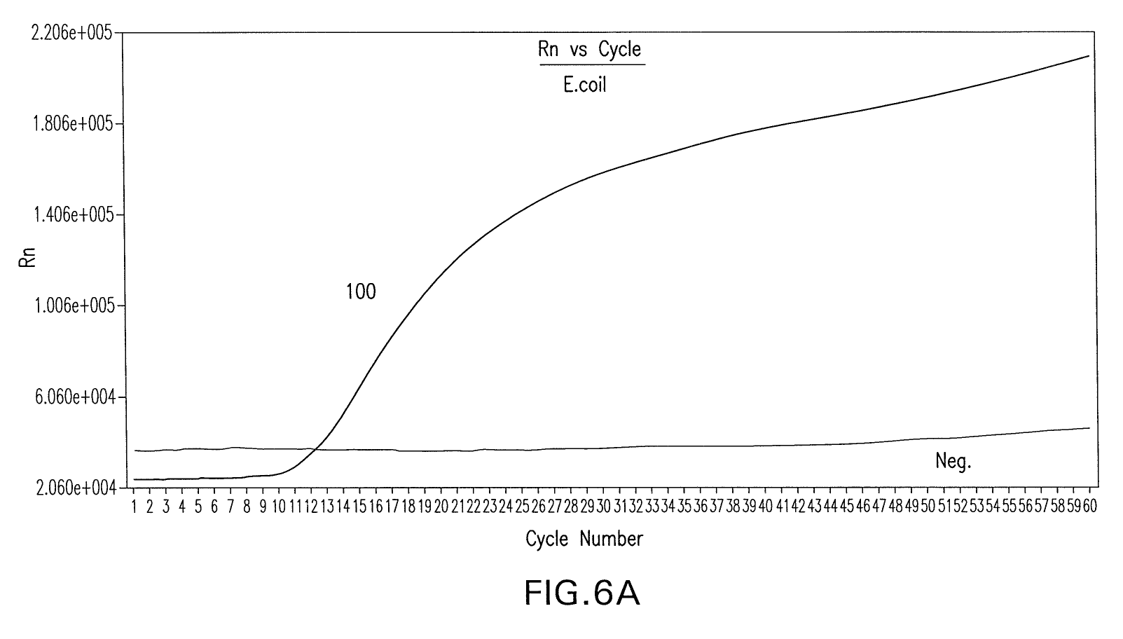

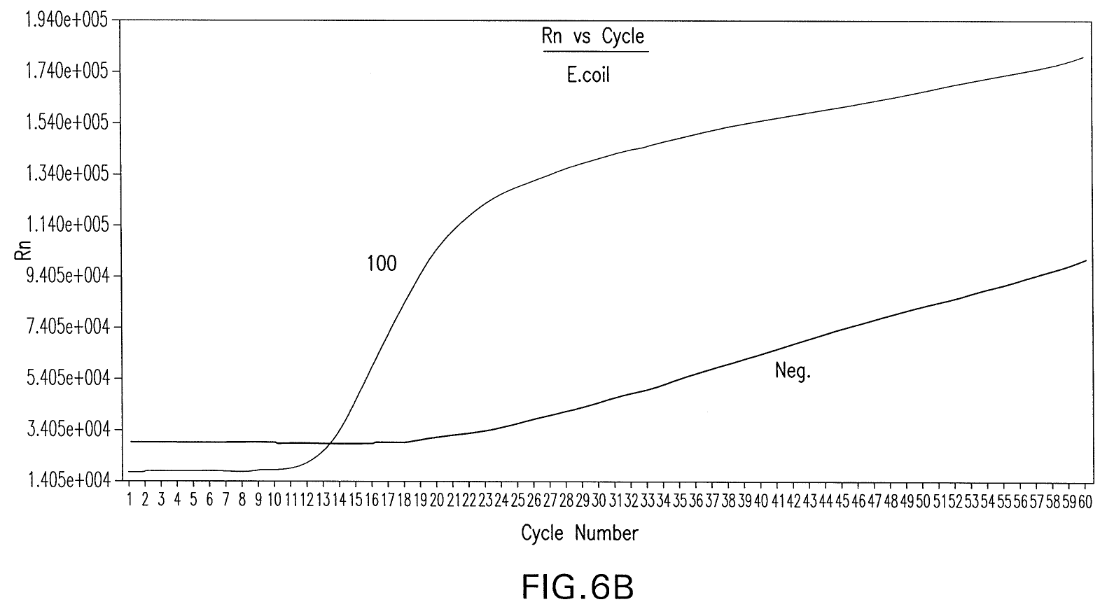

FIG. 6A shows the results using protocol described in this invention when either 100 (100 cells) or 0 (Neg) E. coli cells were added into target capture reagent. Note that no amplification were observed when 0 cells were added into the TCR reagent.

FIG. 6B shows the results using protocol from an existing technology when either 100 (100 cells) or 0 (Neg) E. coli cells were added into target capture reagent. Note that obvious amplification signal was observed when 0 cells were added into the TCR reagent.

As is seen from this example, lower background signal in negative control was observed by using the current invention.

An inherent problem associated with the method in existing technology is that the E. coli-derived nucleic acids contributed by the amplification reagents, or any non-specific amplification using the E. coli-derived nucleic acids contributed by the amplification reagents as templates, can generate fluorescent signal. This is because the detection probe sequence is included in the E. coli nucleic acid sequence, or its complementary.

Instead, with the current invention, one can design a signal primer and a probe in which the signal sequence (which is also used in the probe) doesn't exist in any exogenous/contaminating nucleic acid that may be present in amplification reagents. This design eliminates the background signal that may arise from the binding the probe to the exogenous nucleic acids, or any non-specific amplification products generated from using those exogenous nucleic acids as templates.

### 6.4 EXAMPLE 4

### Simulated Blood Bank Screening for Viruses

6.4.1 Materials and Methods

The target nucleic acids in this examples are that of HIV, HBV and HCV. This example describes a multiplex amplification of HCV, HIV, HBV by real-time transcription mediated amplification (TMA) technology using the current invention. To simplify the overall design, the HIV non-promoter primer is used as the tag primer, and the HIV molecular beacon sequence is used as the “signal sequence” for the design of signal primer for both HBV and HCV amplification and detection.

All of primers, probes, target sequence and the amount used per reaction are listed below, and amplification and detection steps based on those illustrated in FIG. 2.

Oligonucleotides Used:

Signal Primer for HBV (including TAG sequence): TCACAATTTTAAAAGAAAAGGG-A- [symbol] [symbol] -CTGCT TTGCCTCTTCTTC (SEQ ID NO:17); 10 pmol/rxn, the underlined is the signal sequence.

Opposite Primer for HBV: [symbol] [symbol] [symbol] [symbol] -GATAAAACGCCGCAGACACATC (SEQ ID NO:18); 7.5 pmol/rxn, the underlined is the promoter sequence.

Block Oligonucleotide for HBV:

(SEQ ID NO: 19)

uguccugguuaucgcuggaugug; 0.4 pmol/rxn.
Capture oligonucleotide for HBV:

(SEQ ID NO: 20)

gugucuuggccaaaauucgcagucc-
AAAAAAAAAAAAAAAAAAAAAAAAAAAAAAAA; 15 pmol/rxn.

Signal primer for HCV (including TAG sequence): TCACAATTTTAAAAGAAAAGGGA- [symbol] [symbol] -CGGTTCCGCA GACCACTATG (SEQ ID NO:21); 10 pmol/rxn, the underlined is the signal sequence.

Opposite Primer for HCV:

(SEQ ID NO: 22)

[symbol]

[symbol]

[symbol]

[symbol]

-
TCTAGCCATGGCGTTAGTATGAG; 7.5 pmol/rxn, the under-
lined is the promoter sequence.
Block Oligonucleotide for HCV:

(SEQ ID NO: 23)

auggcuagacgcuuucugcgugaag, 0.4 pmol/rxn.
Capture oligonucleotide for HCV:

(SEQ ID NO: 24)

gggcacucgcaagcacccu-
TTTAAAAAAAAAAAAAAAAAAAAAAAAAAAAAA; 15 pmol/rxn.
TAG sequence (HIV non-T7 primer):

(SEQ ID NO: 25)

TCACAATTTTAAAAGAAAAGGG; 7.5 pmol/rxn.
Opposite Primer for HIV:

(SEQ ID NO: 26)

[symbol]

[symbol]

[symbol]

[symbol]

GTTTGTATGTCTGTTGCTATTATGTCTA; 7.5 pmol/rxn.
Capture oligonucleotide for HIV:

(SEQ ID NO: 27)

ucugcugucccuguaauaaacccg-
TTTAAAAAAAAAAAAAAAAAAAAAAAAAAAAAA; 15 pmol/rxn.
Detection Probe (HIV probe):

(SEQ ID NO: 28)

5′-FAM-ccgag- [symbol] [symbol] -cucgg-Dabyl-3′, 5
pmol/rxn.
HCV target sequence:

(SEQ ID NO: 29)

CACTCCACCATGATCACTCCCCTGTGAGGAACTACTGTCTTCACGCAGAA
AGCGTCTAGCCATGGCGTTAGTATGAGTGTTGTACAGCCTCCAGGCCCCC
CCCTCCCGGGAGAGCCATAGTG GTCTGCGGAACCGGTGAGTACACCG
GAATTGCCAGGATGACCGGGTCCTTTCTTGGATTAACCCCGCTCAATGCC
TGGAGATTTGGGCGTGCCCCCGCGAGACTGCTAGCCGAGTAGTGTTGGGT
CGCGAAAGGCCTTGTGGTACTGCCTGATAGGGTGCTTGCGAGTGCCCCGG
GAGGTCTCGTAGACCGTGCATCATGAGCACACTTCCAAAA
HBV target sequence:

(SEQ ID NO: 30)

GAAAGCCCTGCGAACCACTGAACAAATGGCACTAGTAAACTGAGCCAGGA
GAAACGGACTGAGGCCCACTCCCATAGGAATTTTGCGAAAGCCCAGGATG
ATGGGATGGGAATACAAGTGCAGTTTCCGTCCGAAGGTTTTGTACAGCAA
CAAGAGGGAAACATAGAGGTTCCTTGAGCAGGAATCGTGCAGGTCTTGCA
TGGTCCCGTGCTGGTAGTTGATGTTCCTGGAAGTAGAGGACAAACGGGCA
ACATACCTTGGTAGTCCAGAAGAACCAACAAGAAGATGAGGCATAGCAGC
AGGATGAAGAGGAATATGATAAAACGCCGCAGACACATCCA
HIV target sequence.

(SEQ ID NO: 31)

GCCAGTGGATATATAGAAGCAGAAGTTATCCCAGCAGAAACAGGACAGGA
AACAGCATACTTTCTGCTAAAATTAGCAGGAAGATGGCCAGTAAAAGTAA
TACACACAGACAATGGTAGCAATTTCACCAGCAATGCAGTTAAAGCAGCT
TGTTGGTGGGCCAATGTCCGACAGGAATTTGGGATCCCCTACAATCCTCA
AAGTCAAGGAGTAGTAGAATCTATGAATAAGGAATTAAAGAAAATCATAG
GGCAGATAAGAGAACAAGCTGAACACCTTAAGACAGCTGTACAAATGGCA
GTATTCATTCACAATTTTAAAAGAAAAGGGGGGAT TGGGGGGTACAGTG
C
AGGGGAAAGAATAATAGACATAATAGCAACAGACATACAAACTAAAGAAT
TACAAAAACAAATTACAAAAATTCAAAATTTTCGGGTTTATTACAGGGAC
AGCAGAGACCC

Reagents and protocol conditions used in the performed experiments are outlined below.

The TAG primer used in this example is at 15 pmol/rxn. The signal primer is at 15 pmol/rxn. All promoter primers were used at 7.5 pmol/rxn each.

Capture oligonucleotide (also used as blocking oligonucleotide for reverse transcription) is 2′-OME RNA oligonucleotide. Magnetic beads are used as solid phase.

Detection Probe were dual labeled 2′-OME oligonucleotide molecular beacon (5′ Label: 6-Carboxyfluorescein (FAM), 3′ Label: 4-(4′-dimethylaminophenylazo)benzoic acid (DABCYL) and it is used at 5 pmol/rxn.

Amplification reagent contain 26 mM Trizma base buffer, 25 mM MgCl₂, 23.3 mM KCl₂, 3.33% (v/v) glycerol, 0.05 mM zinc acetate, 1.0 mM dATP, 1.0 mM dCTP, 1.0 mM dGTP, 1.0 mM dTTP, 0.02% (v/v), 4.0 mM ATP, 4.0 mM CTP, 4.0 mM GTP, and 4.0 mM UTP, pH 7.81 to 8.0 at 22° C.

Enzyme Reagent contained of 70 mM N-acetyl-L-cysteine, 10% (v/v) TRITON X-102 detergent, 16 mM HEPES, 3 mM EDTA, 20 mM Trizma base buffer, 50 mM KCl₂, 20% (v/v) glycerol, 165.6 mM trehalose, pH 7, and containing 250 RTU/μL Moloney murine leukemia virus (“MMLV”) reverse transcriptase and 140 U/μL T7 RNA polymerase, where one unit of activity is defined as the synthesis and release of 5.75 fmol cDNA in 15 minutes at 37° C. for MMLV reverse transcriptase, and the production of 5.0 fmol RNA transcript in 20 minutes at 37° C. for T7 RNA polymerase.

Wash Buffer contained 10 mM HEPES, 6.5 mM NaOH, 1 mM EDTA, 0.3% (v/v) ethyl alcohol, 1.50 mM NaCl, and 0.1% (w/v) sodium dodecyl sulfate, pH 7.5.

Lyses buffer/target capture reagent contained 150 mM HEPES, 8% (w/v) lithium lauryl sulfate, and 100 mM ammonium sulfate, pH 7.5, 1 micron, super-paramagnetic particles covalently bound 5′-amino modified oligo(dT)₂₅. and all of the capture oligonucleotides, target nucleic acids as described below, and all 3 signal primers.

Amplification reagent also contained all the promoter primers, TAG primer, and the molecular beacon detection probe.

Outline of Protocol:

The lysis buffer and target polynucleotides were mixed and incubated at 60° C. for 10 minutes. After 10 minutes, the magnetic beads can be washed twice with 1 ml wash buffer and the magnetic beads was collected for following steps.

Real-time TMA was performed as below: 30 μl amplification buffer were used to suspend the washed magnetic beads from previous steps, and the mixture was incubated for 5 minutes at 42° C. and then removed and placed on a 42° C. thermomixer. 10 μL aliquot of the Enzyme Reagent was add into each reaction. Shaken gently for 30 seconds on the thermomixer, the reaction mixture was placed into the real-time instrument at 42° C., where real-time assay monitoring was commenced. dTime values, which served as indicators of the amount of amplicon synthesized, was determined from the monitored fluorescent.

6.4.2 Results

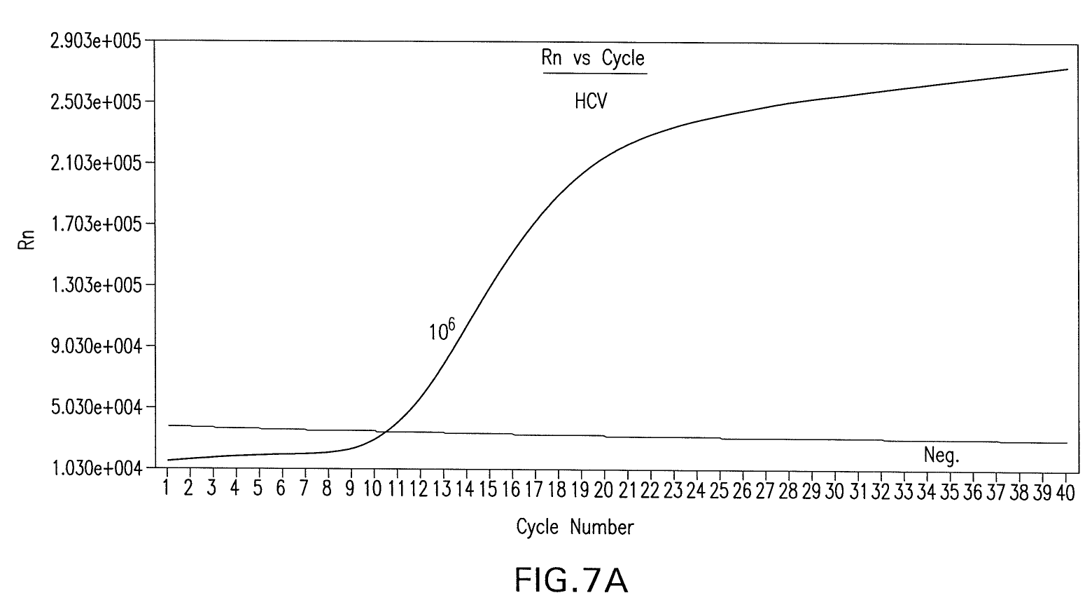

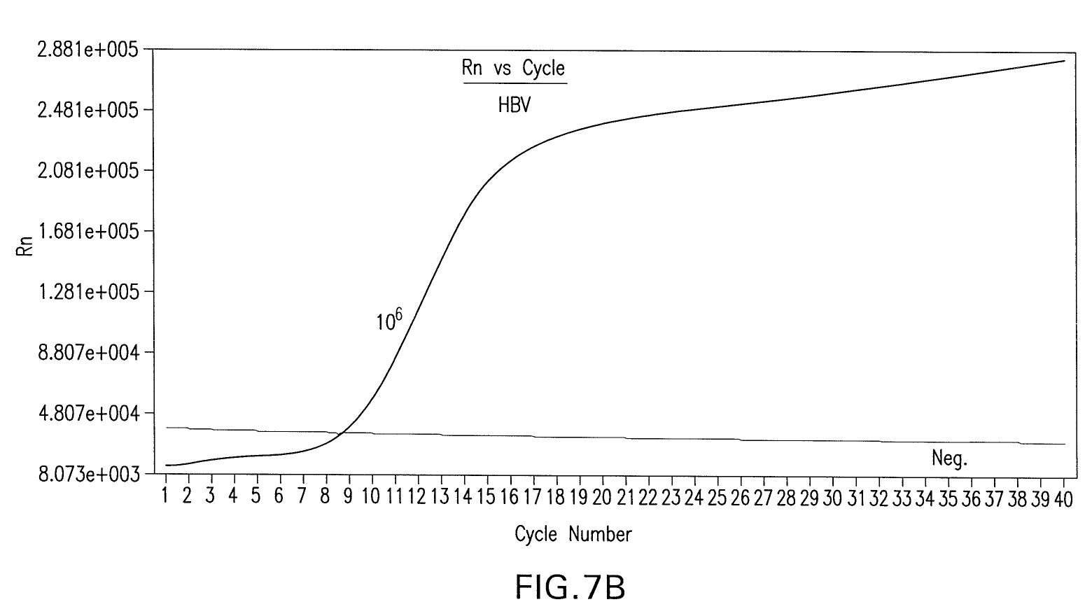

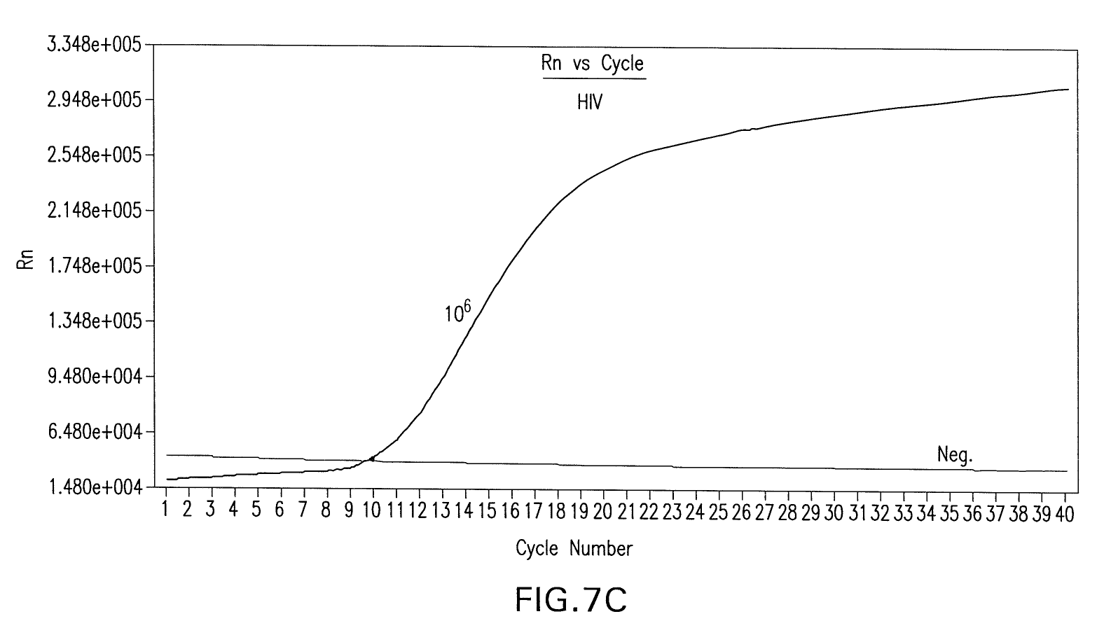

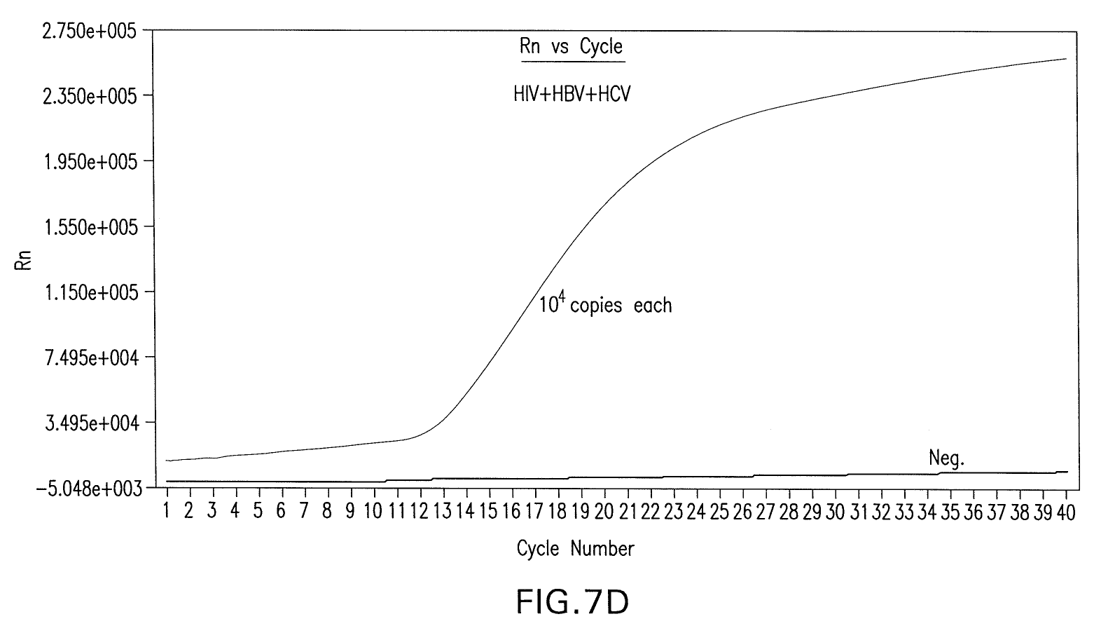

FIG. 7A shows the raw curve of amplificaiton when 10⁶ copies of HCV in vitro transcripts were added into target capture reagent. When no RNA were added (Neg), no amplification were observed.

FIG. 7B shows the raw curve of amplificaiton when 10⁶ copies of HBV in vitro transcripts were added into target capture reagent. When no RNA were added (Neg), no amplification were observed.

FIG. 7C shows the raw curve of amplificaiton when 10⁶ copies of HIV in vitro transcripts were added into target capture reagent. When no RNA were added (Neg), no amplification were observed.

FIG. 7D shows the raw curve of amplificaiton when 10⁴ copies of HCV, HBV, HIV in vitro transcripts were added into target capture reagent. When no RNA were added (Neg), no amplification were observed. In this figure, since only one detection probe was used, if the sample contained more than one target, the experiment would not be able to distinguish which target(s) are present in the sample. In contrast, existing blood screening assays, including GenProbe's PROCLEIX® ULTR10 ® Assay and Roche's COBAS AmpliScreen HIV-1/HCV/HBV Tests, require the use of a target-specific detection probe for each target. Both GenProbe's PROCLEIX® ULTR10 ® Assay and Roche's COBAS AmpliScreen HIV-1/HCV/HBV Tests detects HIV-1, HCV and HBV in donated blood.

### 7. SPECIFIC ASPECTS/EMBODIMENTS, CITATION OF REFERENCES

The present invention is not to be limited in scope by the specific aspects and embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

What is claimed is:

1. A kit for selective amplification and detection of at least one nucleic acid target, said kit comprising one or more container means that comprises:
(a) a signal primer that comprises
(i) a first target-specific (TS1) sequence which hybridizes specifically to a nucleic acid target, and
(ii) a signal (Sg) sequence upstream of the TS1 sequence, wherein the signal sequence is not found in the nucleic acid target or its complementary sequence; and
(b) a capture moiety that comprises a solid support linked with a second target-specific (TS2) sequence which hybridizes specifically to the nucleic acid target, wherein the TS1 and TS2 sequences hybridize to non-overlapping regions of the nucleic acid target; and
(c) an opposite primer that comprises a third target-specific (TS3) sequence selected from a region of the nucleic acid target that is upstream of the region to which the TS1 sequence hybridizes; and
(d) a detection means for detecting the presence of the complementary sequence to the signal sequence.

2. The kit of claim 1, wherein the opposite primer further comprises a promoter (Pm) sequence upstream of the TS3 sequence.

3. The kit of claim 1, which comprises, for each different nucleic acid target, a different set of signal primer, capture moiety, and opposite primer.

4. The kit of claim 1, further comprising a TAG primer which comprises a TAG sequence, wherein the TAG primer does not comprise the TS1 sequence or the signal sequence and the TAG sequence is not found in the signal sequence, the nucleic acid target, or their respective complementary sequences, and the signal primer further comprises the TAG sequence upstream of the signal sequence.

5. The kit of claim 1, wherein each of the signal primer, the capture moiety, the opposite primer and the detection means is placed in separate container means.

6. The kit of claim 1, wherein the signal primer and the capture moiety are placed in a first container means, and the opposite primer and the detection means are placed in one or two container means different from the first container means.

7. The kit of claim 1, wherein the capture moiety comprises (i) the solid support which is attached with a linker oligonucleotide, and (ii) a capture oligonucleotide comprising the TS2 sequence, and, downstream from the TS2 sequence, the complementary sequence to the linker oligonucleotide.

8. The kit of claim 7, wherein the linker oligonucleotide comprises a poly(B) tail, wherein B is a nucleoside.

9. The kit of claim 1, wherein the solid support is selected from the group consisting of nitrocellulose, nylon, glass, polyacrylate, mixed polymers, polystyrene, silane polypropylene, magnetic particle, microtiter plate, column, fiber, and capillary.

10. The kit of claim 1, wherein the detection means comprises the signal sequence.

11. The kit of claim 1, wherein the detection means is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

12. A composition comprising:
(a) a signal primer that comprises
(i) a first target-specific (TS1) sequence which hybridizes specifically to a nucleic acid target, and
(ii) a signal (Sg) sequence upstream of the TS1 sequence, wherein the signal sequence is not found in the nucleic acid target or its complementary sequence; and
(b) a detection means for detecting the presence of the complementary sequence to the signal sequence.

13. The composition of claim 12, wherein the signal primer further comprises, upstream of the signal sequence, a TAG sequence which is not found in the signal sequence, the nucleic acid target, or their respective complementary sequences.

14. The composition of claim 12, wherein the detection means comprises the signal sequence.

15. The composition of claim 12, wherein the detection means is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

16. The composition of claim 12, further comprising a single-stranded RNA comprising, in the direction from the 5′-end to the 3′-end, a region of the nucleic acid target, a complementary sequence to the TS1 sequence, and a complementary sequence to the signal sequence.

17. The composition of claim 12, further comprising a double-stranded DNA comprising, in the direction from the 5′-end to the 3′-end, a region of the nucleic acid target, a complementary sequence to the TS1 sequence, and a complementary sequence to the signal sequence.

18. A method for selective amplification and detection of at least one nucleic acid target, comprising the steps of:
(a) mixing a sample comprising or is suspected of comprising a nucleic acid target with a signal primer and a capture moiety, wherein the signal primer comprises
(i) a first target-specific (TS1) sequence which hybridizes specifically to the nucleic acid target, and
(ii) a signal (Sg) sequence upstream of the TS1 sequence, wherein the signal sequence is not found in the nucleic acid target or its complementary sequence, and
(iii) a TAG sequence upstream of the signal sequence, wherein the TAG sequence is not found in the signal sequence, the nucleic acid target, or their respective complementary sequences; and
the capture moiety comprises a solid support linked with a second target-specific (TS2) sequence which hybridizes specifically to the nucleic acid target, wherein the TS1 and TS2 sequences hybridize to non-overlapping regions of the nucleic acid target;
wherein said mixing is conducted under a condition for the TS1 and TS2 sequences to hybridize to the nucleic acid target and form a complex comprising the nucleic acid target hybridized with the signal primer and the capture moiety;
(b) isolating or purifying the complex to separate it from excess signal primer not hybridized to the nucleic acid target;
(c) mixing the isolated or purified complex with an opposite primer and a TAG primer in a mixture, wherein the TAG primer comprises the TAG sequence, and the opposite primer comprises (i) a third target-specific (TS3) sequence selected from a region of the nucleic acid target that is upstream of the region to which the TS1 sequence hybridizes, and (ii) a promoter (Pm) sequence upstream of the TS3 sequence;
(d) amplifying the nucleic acid target present in Step (c) to generate a plurality of an amplified RNA product that comprises, in the direction from the 5′-end to the 3′-end, the TS3 sequence, the complementary sequence to the TS1 sequence, the complementary sequence to the signal sequence, and the complementary sequence to the TAG sequence; and
(e) detecting the presence of the amplified RNA product with a detection means which is capable of detecting the presence of the complementary sequence to the signal sequence, wherein the detection means is introduced into the mixture during or after Step (c).

19. The method of claim 18, which uses for each different nucleic acid target, a different set of signal primer, capture moiety, and opposite primer.

20. The method of claim 18, wherein the TAG primer does not comprise the TS1 sequence or the signal sequence.

21. The method of claim 18, wherein the capture moiety comprises (i) the solid support which is attached with a linker oligonucleotide, and (ii) a capture oligonucleotide comprising the TS2 sequence, and, downstream from the TS2 sequence, the complementary sequence to the linker oligonucleotide.

22. The method of claim 21, wherein the linker oligonucleotide comprises a poly(B) tail, wherein B is a nucleoside.

23. The method of claim 18, wherein the solid support is selected from the group consisting of nitrocellulose, nylon, glass, polyacrylate, mixed polymers, polystyrene, silane polypropylene, magnetic particle, microtiter plate, column, fiber, and capillary.

24. The method of claim 18, wherein the detection means comprises the signal sequence.

25. The method of claim 18, wherein the detection means is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.

26. A method for selective amplification and detection of a nucleic acid target, comprising the steps of:
(a) mixing a sample comprising or is suspected of comprising a nucleic acid target with a signal primer and a capture moiety, wherein the signal primer comprises
(i) a first target-specific (TS1) sequence which hybridizes specifically to the nucleic acid target, and
(ii) a signal (Sg) sequence upstream of the TS1 sequence, wherein the signal sequence is not found in the nucleic acid target or its complementary sequence, and
(iii) a TAG sequence upstream of the signal sequence, wherein the TAG sequence is not found in the signal sequence, the nucleic acid target, or their respective complementary sequences; and
the capture moiety comprises a solid support linked with a second target-specific (TS2) sequence which hybridizes specifically to the nucleic acid target, wherein the TS1 and TS2 sequences hybridize to non-overlapping regions of the nucleic acid target;
wherein said mixing is conducted under a condition for the TS1 and TS2 sequences to hybridize to the nucleic acid target and form a complex comprising the nucleic acid target hybridized with the signal primer and the capture moiety;
(b) isolating or purifying the complex to separate it from excess signal primer not hybridized to the nucleic acid target;
(c) mixing the isolated or purified complex with an opposite primer and a TAG primer in a mixture, wherein the TAG primer comprises the TAG sequence, and the opposite primer comprises a third target-specific (TS3) sequence selected from a region of the nucleic acid target that is upstream of the region to which the TS1 sequence hybridizes;
(d) amplifying the nucleic acid target present in Step (c) to generate a plurality of an amplified DNA product that comprises a strand, in the direction from the 5′-end to the 3′-end, the TS3 sequence, the complementary sequence to the TS1 sequence, the complementary sequence to the signal sequence, and the complementary sequence to the TAG sequence; and
(e) detecting the presence of the amplified DNA product with a detection means which is capable of detecting the presence of the complementary sequence to the signal sequence, wherein the detection means is introduced into the mixture during or after Step (c).

27. The method of claim 26, wherein the opposite primer further comprises a promoter (Pm) sequence upstream of the TS3 sequence.

28. The method of claim 26, which uses for each different nucleic acid target, a different set of signal primer, capture moiety, and opposite primer.

29. The method of claim 26, wherein the TAG primer does not comprise the TS1 sequence or the signal sequence.

30. The method of claim 26, wherein the capture moiety comprises (i) the solid support which is attached with a linker oligonucleotide, and (ii) a capture oligonucleotide comprising the TS2 sequence, and, downstream from the TS2 sequence, the complementary sequence to the linker oligonucleotide.

31. The method of claim 30, wherein the linker oligonucleotide comprises a poly(B) tail, wherein B is a nucleoside.

32. The method of claim 26, wherein the solid support is selected from the group consisting of nitrocellulose, nylon, glass, polyacrylate, mixed polymers, polystyrene, silane polypropylene, magnetic particle, microtiter plate, column, fiber, and capillary.

33. The method of claim 26, wherein the detection means comprises the signal sequence.

34. The method of claim 26, wherein the detection means is selected from the group consisting of molecular beacon, TaqMan probe, fluorescence resonance energy transfer (FRET) probe, induced FRET (iFRET) probes, minor grove binder (MGB) probe, molecular torch, and hybridization switch probe.


SEQUENCE LISTING (31 sequences)

SEQ ID NO: 1 - DNA, 60 bp, Artificial Sequence
  Signal Primer (including a TAG sequence, a signal sequence, and a TS1 sequence)
tcacaattttaaaagaaaagggtggtgggtacagtgctactacactctaggtttacagtt

SEQ ID NO: 2 - DNA, 22 bp, Artificial Sequence
  TAG Primer
tcacaattttaaaagaaaaggg

SEQ ID NO: 3 - DNA, 55 bp, Artificial Sequence
  Opposite Primer (including a promoter sequence and a TS3 sequence)
aatttaatacgactcactatagggagagagcgcaggcgggtttgtaagtttggta

SEQ ID NO: 4 - RNA, 20 nt, Artificial Sequence
  Block Oligonucleotide
cgcucguuuuacgcccagua

SEQ ID NO: 5 - DNA, 53 bp, Artificial Sequence
  Capture Oligonucleotide (including a TS2 sequence)
guauuaccgcggcugcuggctttaaaaaaaaaaaaaaaaaaaaaaaaaaaaaa

SEQ ID NO: 6 - RNA, 24 nt, Artificial Sequence
  Detection Probe (molecular beacon, including a signal sequence)
ccgagugguggguacagugcucgg

SEQ ID NO: 7 - DNA, 65 bp, Artificial Sequence
  Signal Primer (including a TAG sequence, a signal sequence, and a TS1 sequence)
tcacaattttaaaagaaaagggtggtgggtacagtgcatgtcaagagtaggtaaggttcttcgcg

SEQ ID NO: 8 - DNA, 43 bp, Artificial Sequence
  Opposite Primer (including a promoter oligonucleotide and a TS3 sequence)
aatttaatacgactcactatagggagaaaangaattgacgngg

SEQ ID NO: 9 - DNA, 16 bp, Artificial Sequence
  TS3 Sequence - when present in a probe can be used in to detect a group of bacteria
aaangaattgacgngg

SEQ ID NO: 10 - RNA, 20 nt, Artificial Sequence
  Block Oligonucleotide
cauuugaguuuuaaccuugc

SEQ ID NO: 11 - DNA, 53 bp, Artificial Sequence
  Capture Oligonucleotide (including a TS2 sequence)
ccaggcggucgacuuaacgctttaaaaaaaaaaaaaaaaaaaaaaaaaaaaaa

SEQ ID NO: 12 - DNA, 46 bp, Artificial Sequence
  Detection Probe (molecular beacon, including asignal sequence)
aatttaatacgactcactatagggagacgcaaggttaaaactcaaa

SEQ ID NO: 13 - RNA, 21 nt, Artificial Sequence
  Signal Primer (including a TAG sequence, a signal sequence, and a TS1 sequence)
uugcggccguacuccccaagg

SEQ ID NO: 14 - DNA, 53 bp, Artificial Sequence
  Capture Oligonucleotide (including a TS2 sequence)
uagcuccggaagccacgccutttaaaaaaaaaaaaaaaaaaaaaaaaaaaaaa

SEQ ID NO: 15 - RNA, 21 nt, Artificial Sequence
  Alternate Detection Probe
ccgagccccgucaauucucgg

SEQ ID NO: 16 - DNA, 50 bp, Artificial Sequence
  Alternate Primer
tcacaattttaaaagaaaagggatgtcaagagtaggtaaggttcttcgcg

SEQ ID NO: 17 - DNA, 57 bp, Artificial Sequence
  Signal Primer for HBV (including a TAG sequence)
tcacaattttaaaagaaaagggagtggagggtacagtgcctgctttgcctcttcttc

SEQ ID NO: 18 - DNA, 49 bp, Artificial Sequence
  Opposite Primer for HBV
aatttaatacgactcactatagggagagataaaacgccgcagacacatc

SEQ ID NO: 19 - RNA, 23 nt, Artificial Sequence
  Block Oligonucleotide for HBV
uguccugguuaucgcuggaugug

SEQ ID NO: 20 - RNA, 57 nt, Artificial Sequence
  Capture Oligonucleotide for HBV
gugucuuggccaaaauucgcaguccaaaaaaaaaaaaaaaaaaaaaaaaaaaaaaaa

SEQ ID NO: 21 - DNA, 59 bp, Artificial Sequence
  Signal Primer for HCV (including a TAG sequence)
tcacaattttaaaagaaaagggagtggagggtacagtgccggttccgcagaccactatg

SEQ ID NO: 22 - DNA, 50 bp, Artificial Sequence
  Opposite Primer for HCV
aatttaatacgactcactatagggagatctagccatggcgttagtatgag

SEQ ID NO: 23 - RNA, 25 nt, Artificial Sequence
  Block Oligonucleotide for HCV
auggcuagacgcuuucugcgugaag

SEQ ID NO: 24 - DNA, 52 bp, Artificial Sequence
  Capture Oligonucleotide for HCV
gggcacucgcaagcacccutttaaaaaaaaaaaaaaaaaaaaaaaaaaaaaa

SEQ ID NO: 25 - DNA, 22 bp, Artificial Sequence
  TAG Sequence (HIV non-T7 primer)
tcacaattttaaaagaaaaggg

SEQ ID NO: 26 - DNA, 55 bp, Artificial Sequence
  Opposite Primer for HIV
aatttaatacgactcactatagggagagtttgtatgtctgttgctattatgtcta

SEQ ID NO: 27 - DNA, 57 bp, Artificial Sequence
  Capture Oligonucleotide for HIV
ucugcugucccuguaauaaacccgtttaaaaaaaaaaaaaaaaaaaaaaaaaaaaaa

SEQ ID NO: 28 - RNA, 26 nt, Artificial Sequence
  Detection Probe (HIV probe)
ccgagguggaggguacagugccucgg

SEQ ID NO: 29 - DNA, 337 bp, Artificial Sequence
  HCV Target Sequence
cactccaccatgatcactcccctgtgaggaactactgtcttcacgcagaaagcgtctagccatggcgttagtatgagtgttgtacagcctccaggcccccccctcccgggagagccatagtggtctgcggaaccggtgagtacaccggaattgccaggatgaccgggtcctttcttggattaaccccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttccaaaa

SEQ ID NO: 30 - DNA, 341 bp, Artificial Sequence
  HBV Target Sequence
gaaagccctgcgaaccactgaacaaatggcactagtaaactgagccaggagaaacggactgaggcccactcccataggaattttgcgaaagcccaggatgatgggatgggaatacaagtgcagtttccgtccgaaggttttgtacagcaacaagagggaaacatagaggttccttgagcaggaatcgtgcaggtcttgcatggtcccgtgctggtagttgatgttcctggaagtagaggacaaacgggcaacataccttggtagtccagaagaaccaacaagaagatgaggcatagcagcaggatgaagaggaatatgataaaacgccgcagacacatcca

SEQ ID NO: 31 - DNA, 461 bp, Artificial Sequence
  HIV Target Sequence
gccagtggatatatagaagcagaagttatcccagcagaaacaggacaggaaacagcatactttctgctaaaattagcaggaagatggccagtaaaagtaatacacacagacaatggtagcaatttcaccagcaatgcagttaaagcagcttgttggtgggccaatgtccgacaggaatttgggatcccctacaatcctcaaagtcaaggagtagtagaatctatgaataaggaattaaagaaaatcatagggcagataagagaacaagctgaacaccttaagacagctgtacaaatggcagtattcattcacaattttaaaagaaaaggggggattggggggtacagtgcaggggaaagaataatagacataatagcaacagacatacaaactaaagaattacaaaaacaaattacaaaaattcaaaattttcgggtttattacagggacagcagagaccc